SEQUENCE LISTING (17 sequences)

SEQ ID NO: 1 - DNA, 27 bp, Artificial
  PCR primer
gatatccaccatggcttgggtgtggac

SEQ ID NO: 2 - DNA, 30 bp, Artificial
  PCR primer
gatatcttatcatttaccaggagagtggga

SEQ ID NO: 3 - DNA, 31 bp, Artificial
  PCR primer
gatatccaccatggtgtccacttctcagctc

SEQ ID NO: 4 - DNA, 33 bp, Artificial
  PCR primer
gatatcttactaacactcattcctgttgaagct

SEQ ID NO: 5 - DNA, 23 bp, Artificial
  PCR primer
aattccttaagctcgagtcgcga

SEQ ID NO: 6 - DNA, 23 bp, Artificial
  PCR primer
aatttcgcgactcgagcttaagg

SEQ ID NO: 7 - DNA, 26 bp, Artificial
  PCR Primer
ctcgaggatatcgaattcctgcagcc

SEQ ID NO: 8 - DNA, 27 bp, Artificial
  PCR Primer
ctcgagcccgatgataagacgactatg

SEQ ID NO: 9 - DNA, 20 bp, Artificial
  PCR Primer
atgaggcttcgggagccgct

SEQ ID NO: 10 - DNA, 23 bp, Artificial
  PCR Primer
ctagctcggtgtcccgatgtcca

SEQ ID NO: 11 - DNA, 21 bp, Artificial
  PCR Primer
atgaggttccgcatctacaaa

SEQ ID NO: 12 - DNA, 30 bp, Artificial
  PCR Primer
tcactgcagtcttctataacttttacagag

SEQ ID NO: 13 - DNA, 50 bp, Bombyx mori nuclear polyhedrosis virus
aagtattttactgttttcgtaacagttttgtaataaaaaaacctataaat

SEQ ID NO: 14 - DNA, 81 bp, Artificial
  PCR Primer
cacctcgcgaaagtattttactgttttcgtaacagttttgtaataaaaaaacctataaatatgaagtttcgggagccgctc

SEQ ID NO: 15 - DNA, 27 bp, Artificial
  PCR Primer
tcgcgattagctcggcgtcccgatgtc

SEQ ID NO: 16 - DNA, 79 bp, Artificial
  PCR Primer
cacctcgcgaaagtattttactgttttcgtaacagttttgtaataaaaaaacctataaatatgaggttccgcatctaca

SEQ ID NO: 17 - DNA, 37 bp, Artificial
  PCR Primer
tcgcgattactgcagtcttctataacttttacagagt

(19) United States
(12) Patent Application Publication
(10) Pub. No.: US 2011/0203009 A1
(43) Pub. Date: Aug. 18, 2011

(54) METHOD FOR PRODUCING GLYCOPROTEIN CHARACTERIZED BY SUGAR CHAIN STRUCTURE BY USING SILKWORM

(76) Inventors: Masahiro Tomita, Tokyo (JP); Shinichi Nakakita, (US); Jun Hirabayashi, Kanagawa (JP)

(21) Appl. No.: 12/997,333

(22) PCT Filed: Jun. 15, 2009
(86) PCT No.: PCT/JP2009/002715

§ 371 Date: Dec. 10, 2010

(30) Foreign Application Priority Data
Jun. 13, 2008 (JP) 2008-155901

(51) Int. Cl. C12P 21/00; C12N 5/10; A01K 67/033; C07H 21/04

(52) U.S. Cl. 800/4; 435/70.3; 435/69.1; 435/348; 800/13; 536/23.2

(57) ABSTRACT

It is an object of the present invention to investigate what kinds of structures a sugar chain has by performing a sugar chain structural analysis of a glycoprotein produced in a transgenic silkworm. For the sugar chain of the glycoprotein produced in the transgenic silkworm, a glycoprotein having a sugar chain structure in which fucose is not linked to N-acetylglucosamine at a reducing terminal of the sugar chain is obtained. In addition, a glycoprotein having an almost completely humanized sugar chain structure is obtained by further integrating GalT gene into this transgenic silkworm.

### BACKGROUND OF THE INVENTION

1. Fields of the Invention

The present invention relates to a method for producing a glycoprotein characterized by a sugar chain structure.

2. Description of the Related Art

Recently, production of a recombinant protein by use of E. coli, etc. has started to be applied to production of a glycoprotein such as an antibody and a blood protein, which has the effect only when it is used in a relatively large amount, in addition to production of a cytokine and a hormone, which has the effect even when it is used in a small amount. Since E. coli cannot intrinsically synthesize a glycoprotein, many pharmaceutical companies are conducting the production by using a mammalian cultured cell. When the mammalian cultured cell is used, the large scale production is difficult, and the higher cost cannot be avoided. Due to them, there has been a demand for an expression system having a large scale and economical production of a glycoprotein pharmaceutical.

Neosilk Corp., Ltd. and Hiroshima Prefectural Institute of Industrial Science and Technology have succeeded in creating a transgenic silkworm (Bombyx mori) which secretes a recombinant protein as a component of silk thread (Patent Documents 1 to 7), and have carried out a contract production of the recombinant protein and have developed a protein pharmaceutical by using this silkworm.

In contrast, in institutions other than Neosilk Corp. and Hiroshima Prefectural Institute of Industrial Science and Technology, there has been a certain degree of research on the production of the glycoprotein by using an insect cell. However, when the protein is expressed in a baculovirus-insect cell expression system, most of the N-linked sugar chain is a trimannosyl-core type as shown in pathway 1 of FIG. 7. This is due to lack of an activity of galactosyltransferase (GalT) and sialyltransferase (ST) in the insect cell, which is a host. Furthermore, in the insect cell host, this is also due to the fact that N-acetylglucosaminidase (GlcNAcase), which is an enzyme localized in Golgi apparatus, has an activity to construct the insect-type N-linked sugar chain added to a glycoprotein expressed in the insect cell. That is, it is considered that the enzyme prohibits elongation of the sugar chain and elimination of α-1,3-fucose by hydrolyzing an acetylglucosamine residue at the N-linked sugar chain terminal of the synthesis pathway. In contrast, a complex type (a mammalian type) sugar chain that mammalian-derived glycoproteins possess is synthesized by pathway 2 of FIG. 7, and sialic acid is added to its terminal.

Because of this, there raises a problem that the insect-type sugar chain is recognized as a foreign substance in vivo in mammals, or that a recombinant glycoprotein cannot stay for a long period in vivo when it is administered to the mammals. Therefore, if the mammal-derived glycoprotein is expressed in the insect cells, the stability in the mammals in vivo is low so that it is not suitable for administration to mammals.

In order to provide a method for expressing, in an insect cell, a recombinant glycoprotein which is superior in the stability in vivo, which has a suitable dosage form of administration to mammals, which is similar to a native mammalian protein, and to which sialic acid is added, developed is a method for expressing, in an insect or insect cell, a recombinant glycoprotein having a sugar chain to which sialic acid is add, the method comprising reducing, inhibiting, or removing N-acetylglucosaminidase activity that the insect cell possesses (Patent Document 8).

In addition, there is a research revealing a fucose linkage of an N-linked sugar chain of the glycoprotein expressed in insect cultured cells. According to the research, there is a case in which fucose is added to N-acetylglucosamine at a reducing terminal of the N-linked sugar chain of the glycoprotein via α-1, 3-linkage or α-1,6-linkage (Non-Patent Documents 1 and 2). It is known that the fucose via α-1, 3-linkage (α-1,3-fucose) has antigenicity in vivo in mammals. Accordingly, the glycoprotein produced using the insect cultured cells is considered to be unsuitable for administration to mammals. Additionally, although generation of a silkworm recombinantly incorporating a galactosyltransferase gene has not been reported in patent publications or journal publications, there is a report in a scientific meeting, and there is also a disclosure in a homepage (Non-Patent Document 3).

On the other hand, for clinical applications of an anti-tumor antibody, there are currently many cases in which sole use of the antibody is insufficient for anti-tumor effects. Thus, current practice utilizes combination therapy with chemotherapy. However, if more potent anti-tumor effects of the antibody alone due to an increase in an antibody-dependent cellular cytotoxic activity (ADCC activity) are observed, the dependency on the chemotherapy decreases, and its side effects should be reduced. Then, Kyowa Hakko Kogyo Co., Ltd. has specified a sugar chain increasing the ADCC activity by analyzing the sugar chain of the antibody belonging to human IgG1 subclass, which is produced in a variety of animal cells. As a purpose to provide a method for modulating an activity of an immunofunctional molecule, the ADCC activity of a humanized antibody produced in rat myeloma YB2/0 cells is found to be markedly higher than humanized antibodies produced in other cells (Patent Document 9).

Based on this discovery, Kyowa Hakko Kogyo Co., Ltd. has further developed the research, and by way of BioWa, Inc., a U.S. subsidiary of Kyowa Hakko Kogyo, the company has been conducting a business such as licensing to worldwide pharmaceutical companies the POTELLIGENT® technology by which the ADCC activity is enhanced (Patent Documents 10 to 18).

### RELATED DOCUMENTS

### Patent Documents

[Patent Document 1] JP-A-2001-161214
[Patent Document 2] JP-A-2002-306167
[Patent Document 3] JP-A-2002-315580
[Patent Document 4] JP-A-2004-16144
[Patent Document 5] JP-A-2004-344123
[Patent Document 6] JP-A-2006-16323
[Patent Document 7] JP-A-2006-109772
[Patent Document 8] JP-A-2003-70469
[Patent Document 9] WO 00/61739
[Patent Document 10] WO 02/031140
[Patent Document 11] WO 03/055993
[Patent Document 12] WO 03/084569
[Patent Document 13] WO 03/084570
[Patent Document 14] WO 03/085102
[Patent Document 15] WO 03/085118
[Patent Document 16] WO 03/085119
[Patent Document 17] WO 05/053742
[Patent Document 18] JP-A-2007-129903

### Non-Patent Documents

[Non-patent Document 1] Kubelka, V. et al., Arch. Biochem Biophys. 308, 148-157, 1994
[Non-patent Document 2] Staudacher, E. et al., Eur. J. Biochem. 207, 987-993, 1992
[Non-Patent Document 3] Mori, H., “Development of a system for producing a useful substance by transformation of a silkworm”, http://www.jst.go.jp/shincho/db/seika/2006_s/2006_s_{—}9/2006_s_{—}9_koncyukinou/2006_s_{—}9_koncyukinou_{—}3_{—}1_{—}3.htm

### SUMMARY OF THE INVENTION

### Problems to be Solved by the Invention

However, the conventional techniques described in the above documents still has room for improvement in the following points.

First, Patent Documents 1 to 7 disclose a success in creating a transgenic silkworm secreting a recombinant protein as a component of silk thread. Previous findings teach that a sugar chain is added to a protein produced in a silkworm, but this structure of the sugar chain is not identical to that of proteins synthesized in mammals. Because of this, this structure of the sugar chain is a problem when a glycoprotein pharmaceutical is produced in a silkworm. In addition, even if this problem is solved by genetic engineering of a silkworm cell or silkworm body, the analysis of the structure of the sugar chain that is added in the silkworm is necessary so as to select the glycosyltransferase that is required to modify the sugar chain.

Second, Patent Document 8 discloses a success in expressing, in an insect or insect cell, a recombinant protein having a sugar chain to which sialic acid is added. However, there is no investigation as to conditions of a fucose linkage in the sugar chain structure of the glycoprotein expressed in a transgenic silkworm. Due to this, when an antibody derived from a transgenic silkworm is produced, it is unclear what kinds of the ADCC activity and antigenicity are observed about the antibody in vivo in mammals.

Third, Patent Documents 9 to 18 disclose a success in specifying a sugar chain that enhances the ADCC activity. An efficient and cost-effective production system is not yet established. That is, since the synthesis of a glycoprotein cannot be performed in E. coli, many companies have produced it by using a mammalian cultured cell. However, when the mammalian cultured cell is used, the large scale production is difficult, and the high cost cannot be avoided.

The present invention has been carried out in view of the above situation. It is an object of the present invention to investigate the structures of the sugar chain by analyzing glycoproteins produced in a transgenic silkworm, including IgG to begin with. In addition, it is another object of the present invention to provide a technique which enables efficient and low-cost production of a glycoprotein characterized by a sugar chain structure by using an insect body or insect cell on the basis of the results obtained by the above sugar chain structural analysis.

### Means to Solve the Problems

The present invention provides a method for producing a glycoprotein, the method comprising the step of expressing, in a silk gland cell of an insect, a glycoprotein having an N-glycoside-linked sugar chain in which fucose is not linked to N-acetylglucosamine at a reducing terminal of the sugar chain.

According to this method for production, since the glycoprotein is expressed in the silk gland cell of the insect, the glycoprotein having the N-glycoside-linked sugar chain in which fucose is not linked to N-acetylglucosamine at the reducing terminal of the sugar chain is expressed efficiently. In addition, since the glycoprotein expressed can also be efficiently extracted, it is possible to produce, efficiently and at low cost, the glycoprotein characterized by the sugar chain structure by using the insect cell.

The present invention provides a method for producing a glycoprotein having an N-glycoside-linked sugar chain in which fucose is not linked to N-acetylglucosamine at a reducing terminal of the sugar chain, the method comprising the step of expressing the glycoprotein in a silk gland localized inside an insect body, a silk gland tissue extirpated from the insect body, or a silk gland cell obtained from the silk gland tissue extirpated from the insect body.

Since this method for production allows the glycoprotein to be expressed in the silk gland localized inside the insect body, the silk gland tissue dissected from the insect body, or the silk gland cell obtained from the silk gland tissue dissected from the insect body, the glycoprotein having the N-glycoside-linked sugar chain in which fucose is not linked to N-acetylglucosamine at the reducing terminal of the sugar chain can be expressed efficiently, and the glycoprotein expressed can be efficiently extracted. Accordingly, the glycoprotein characterized by the sugar chain structure can be produced efficiently and at low cost by using the insect cell.

In addition, the present invention provides a glycoprotein-producing cell, comprising a cell producing a glycoprotein having an N-glycoside-linked sugar chain in which fucose is not linked to N-acetylglucosamine at a reducing terminal of the sugar chain, the cell being an insect silk gland cell genetically modified to express the glycoprotein.

According to this glycoprotein-producing cell, in the insect silk gland cell genetically modified to express the glycoprotein, the glycoprotein having the N-glycoside-linked sugar chain in which fucose is not linked to N-acetylglucosamine at the reducing terminal of the sugar chain can be expressed efficiently so as to express the glycoprotein. In addition, since the glycoprotein expressed can be efficiently extracted, it is possible to produce, efficiently and at low cost, the glycoprotein characterized by the sugar chain structure by using the insect cell.

In addition, the present invention provides a glycoprotein-producing organism producing a glycoprotein having an N-glycoside-linked sugar chain in which fucose is not linked to N-acetylglucosamine at a reducing terminal of the sugar chain, the organism comprising an insect body genetically modified to express the glycoprotein in a silk gland.

According to this glycoprotein-producing organism, in the insect silk gland genetically modified to express the glycoprotein in the silk gland, the glycoprotein having the N-glycoside-linked sugar chain in which fucose is not linked to N-acetylglucosamine at the reducing terminal of the sugar chain can be expressed efficiently so as to express the glycoprotein. In addition, since the glycoprotein expressed can be efficiently extracted, it is possible to produce, efficiently and at low cost, the glycoprotein characterized by the sugar chain structure by using the insect cell.

Besides, the above production methods are one of aspects of the present invention, and a production method of the present invention may be any combinations of the above configurations. Additionally, a glycoprotein-producing tissue of the present invention has a similar configuration, and exerts a similar effect.

### Advantageous Effect of the Invention

According to the present invention, since a particular type of an insect body, insect tissue, or insect cell is used, a glycoprotein characterized by a sugar chain structure can be produced efficiently and at low cost by using the insect body, insect tissue, or insect cell.

### BRIEF DESCRIPTION OF THE DRAWINGS

FIG. 1 is a diagram showing the structure of IgG(H)/pMSG1.1R and IgG(L)/pMSG1.1MG vectors. In the upper part, FIG. 1A is a diagram showing the structure of the vector (IgG(H)/pMSG1.1R) used to generate a transgenic silkworm expressing an H-chain of an antibody in a middle silk gland.

FIG. 1 is a diagram showing the structure of IgG(H)/pMSG1.1R and IgG(L)/pMSG1.1MG vectors. In the lower part, FIG. 1B is a diagram showing the structure of the vector (IgG(L)/pMSG1.1MG) used to generate a transgenic silkworm expressing an L-chain of an antibody.

FIG. 2 is a chart showing results of a mass spectrometry analysis of the N-glycoside-linked sugar chain of IgG.

FIG. 3 is a chart showing results obtained by separating the PA sugar chain prepared from murine IgG expressed in a silkworm by anion exchange HPLC.

FIG. 4 is a chart showing results obtained by separating the resulting neutral sugar chain by size-fractionation HPLC.

FIG. 5 is a chart showing results obtained by separating the resulting sugar chain by reversed-phase HPLC.

FIG. 6 is a diagram of the sugar chain structures in which the sugar chain structure of murine IgG expressed in a silkworm is summarized. The numerical values designated between parentheses in the figure denote the number of moles contained in fraction No. 3 to No.8, and the percentage (%) of the number of moles of the sugar chain in each fraction to the total number of moles.

FIG. 7 is a schematic diagram illustrating a synthesis pathway of an N-linked sugar chain in an insect and a mammal.

FIG. 8 is a diagram of the sugar chain structures in which the sugar chain structures in the middle silk gland of pnd-w1 are listed.

FIG. 9 is a diagram of the sugar chain structures in which the sugar chain structures in the middle silk gland of Kinshu are listed.

FIG. 10 is a diagram of the sugar chain structures in which the sugar chain structures in the cocoon of pnd-w1 are listed.

FIG. 11 is a diagram of the sugar chain structures in which the sugar chain structures in the cocoon of Kinshu are listed.

FIG. 12 is a diagram of the sugar chain structures in which the sugar chain structures in the fat body of pnd-w1 are listed.

FIG. 13 is a diagram of the sugar chain structures in which the sugar chain structures in the fat body of Kinshu are listed.

FIG. 14 is a diagram showing the vector structure of hGalT1-hGnT2/pMSG3.1R vector.

FIG. 15 is a photo of the electrophoresis showing a comparative result of expression levels of hGalT1 in the middle silk gland of GalT-GnT2-TG silkworm.

FIG. 16 is a diagram of the sugar chain structures comparing with the sugar chain structures in the cocoon of GalT-GnT2-TG silkworm. The numerical values in the figure denote the percentage (%) of the number of moles of the sugar chain in each fraction to the total number of moles of the sugar chain.

### DESCRIPTION OF THE PREFERRED EMBODIMENTS

### <Definition of Terms>

The phrase “the lower limit numerical value to the upper limit numerical value” herein means that the value is within the extent of the lower limit numerical value or more and the upper limit numerical value or less.

The term “polynucleotide” herein means a molecule combined with two or more nucleoside phosphoesters (ATP, GTP, CTP, UTP; or dATP, dGTP, dCTP, dTTP) in which purine or pyrimidine is linked to a sugar via a β-N-glycoside linkage. The phrase that one polynucleotide is “operably linked” to another polynucleotide means a preserved state in which a desired function can be exerted by a linkage without impairing the functions of each polynucleotide. Specifically, this refers to a state in which the 3′-end of one polynucleotide is linked to the 5′-end of another polynucleotide directly or via another linker sequence.

The term “protein” herein means a molecule comprising a plurality of amino acid residues linked one another via an amide bond (a peptide bond). The term “recombinant protein” means a protein produced using genetic engineering.

The term “glycoprotein” herein means a typical glycoprotein, and refers to a molecule in which a sugar chain is linked to a portion of amino acid constituting a protein. In animals, most of the proteins expressed on a surface or secreted extracellularly are considered to be a glycoprotein. For modifications of amino acid in the protein, frequently observed are two kinds of modifications: one linked to asparagine (N-linked); and the other linked to serine or threonine (O-linked; mucin-type).

There are not many types of sugars constituting a sugar chain linked to a glycoprotein. Examples of the frequently observed sugar include about seven to eight types such as glucose, galactose, mannose, fucose, N-acetylglucosamine, N-acetylgalactosamine, N-acetylneuramic acid, and xylose. There are limited numbers of structural configurations in a certain extent. Thus, a variety of biological phenomena are regulated by distinguishing a small structural difference among them and by precisely recognizing the difference.

The term “antibody” herein means a typical antibody, which is a glycoprotein molecule produced by B cells among lymphocytes, and functions to recognize and bind to a molecule (antigen) such as a specific protein. In addition, the term “antibody” is a name emphasized on the function in which it binds to an antigen. It is called an immunoglobulin as a substance, and is abbreviated as “Ig”. All antibodies are an immunoglobulin, and fall under γ-globulin in plasma.

The antibodies can be divided into several classes (isotypes) depending on a difference in their constant region. In mammals, due to the difference in the structure of the constant region, the antibodies can be classified into five classes of immunoglobulins, including IgG, IgA, IgM, IgD, and IgE. In the case of human, IgG has four subclasses, including IgG1 to IgG4. IgA possesses two subclasses, including IgA1 and IgA2. Each has a slightly different structure. IgM, IgD, and IgE have no subclass. In addition, the term “antibody” herein means a general term including the above classes and subclasses, may be an antibody fragment such as Fab and F(ab′)2, and may include a murine antibody, a chimeric antibody, a humanized antibody, and a human antibody.

The term “gene encoding a protein” herein refers to a polynucleotide comprising a region encoding a protein (open reading frame: ORF). The examples include a cDNA of the gene encoding the protein. The term “gene promoter region” refers to a region that presents in an upstream region from the transcription start site of the gene region encoding a protein, the upstream region comprising an essential sequence for initiating a transcription. It is generally referred to as a “promoter region” or an “enhancer region”. The term “gene encoding an H-chain of an antibody” or “gene encoding an L-chain of an antibody” refers to a polynucleotide comprising a region encoding an H-chain or an L-chain of an antibody (open reading frame: ORF), respectively. For example, they refer to cDNAs of the genes encoding these chains.

The term “fucose” herein means typical fucose, which is a kind of deoxy sugar, 6-deoxy-galactose. The chemical formula is C₆H₁₂O₅, the molecular weight is 164.16, the melting point is 163° C., and the specific optical rotation is −76 degree. It is classified as a hexose and a monosaccharide. Fucose exists widely in animals and plants as a form of L-type (i.e., L-fucoside) naturally. In mammals and plants, the fucose is found to be in an N-linked sugar chain on a cell surface.

The term “N-acetylglucosamine” herein means typical N-acetylglucosamine (e.g., N-acetyl-D-glucosamine, GlcNAc, NAG), which is a monosaccharide derived from glucose, and is an important substance for several biochemical mechanisms. Chemically, this substance is defined as an amide of glucosamine and acetic acid. In mammals, N-acetylglucosamine is a component of a glycoprotein, hyaluronic acid, and glycosaminoglycan (mucopolysaccharide). N-acetylglucosamine is a primary sugar constituting a sugar chain having a further complicated structure, as well as constitutes a structure of an N-linked glycoprotein in which an oligosaccharide chain primarily composed of mannose is linked to asparagine.

The term “galactose” herein means typical galactose, the molecular structure and the molecular weight of which are C₆H₁₂O₆ and 180, respectively. These characteristics are the same as glucose. For the conformation, —OH of position 2 (located at the second from the top in Fisher projection) and position 5 has the same direction, and that of position 3 and position 4 has an inverted direction. D-galactose has the same orientation as D-glyceraldehyde at position 5. Galactose is a 4-epimer of glucose. In nature, D-galactose accounts for almost all of the galactose.

The term “β-galactosyltransferase” herein means typical β-galactosyltransferase (β-GalT), which is an enzyme forming a structure of Gal-β-1,3/4-GlcNAc by transferring Gal from UDP-Gal to a GlcNAc residue being a terminal of an sugar chain. Previously, the genes encoding β-1,4-GalT and β-1,3-GalT was unknown, but recently they have been cloned one after another. It has been revealed that each of them also form a family. For example, human β-1,4-GalT genes have been cloned using the amino acid sequence homology observed among glycosyltransferases or the sequence information of the gene fragments deposited at a gene bank. Since they have 55%, 44%, 41%, 37%, and 33% homology with the amino acid sequence of β-1,4-GalT I, they are designated as β-1,4-GalT II, III, IV, V, and VI, respectively, according to the order of the homology. The “β-galactosyltransferase” herein includes all of the β-1,4-GalT and β-1,3-GalT.

The term “N-acetylglucosaminyltransferase” herein means typical N-acetylglucosaminyltransferase (GnT), and N-acetylglucosaminyltransferase I, II, III, IV, V, and VI (GnT-I, II, III, IV, V, VI) are known. Among them, in respect to the high branching of the N-linked sugar chain, N-acetylglucosaminyltransferase III, IV, V, and VI (GnT-III, IV, V, VI) determine the core region of the branching structure. Accordingly, the regulation of the activity of these enzymes is considered to result in a change in the overall sugar chain structure. In contrast, N-acetylglucosaminyltransferase I and II (GnT-I, II) are considered to have an important role in determining an essential core structure. The “N-acetylglucosaminyltransferase” herein includes all of the N-acetylglucosaminyltransferase I, II, III, IV, V, and VI.

The term “operably linked DNA sequence” herein refers to, for example, a DNA sequence obtained by operably linking a DNA sequence encoding β-galactosyltransferase to a DNA sequence encoding a sericin promoter. A sequence containing the DNA sequence encoding β-galactosyltransferase is transcribed to synthesize mRNA by the function of the sericin promoter, and the mRNA is translated to produce β-galactosyltransferase. The term also refers to, for example, a DNA sequence obtained by operably linking a DNA sequence encoding N-acetylglucosaminyltransferase to a DNA sequence encoding a sericin promoter. A sequence containing the DNA sequence encoding N-acetylglucosaminyltransferase is transcribed to synthesize mRNA by the function of the sericin promoter, and the mRNA is translated to produce N-acetylglucosaminyltransferase.

The “DNA construct” herein includes a vector derived from a plasmid, a vector derived from a phage, a cosmid vector, an artificial chromosomal vector, a variety of polynucleotides, and the like. Besides, since the handling is convenient and the versatility is high, the vector derived from a plasmid is preferred.

The term “insect” herein means a typical insect, which is a general term of organisms classified into Insecta.

The term “Lepidoptera” herein means typical Lepidoptera, which is one of classifications of insect kinds called “Cho-moku” (Lepidoptera) or “Ga-moku” (Lepidoptera). Since the body surface of an imago is covered with scales and hairs, Japanese translation of Lepidoptera is named after it. In addition, its developmental stages underdo a complete metamorphosis, including an embryo (egg), a larva, a pupa, and an imago. The larva is in a cylindrical shape and has a soft body. The ambulatory legs of the chest are short, and the abdomen has foot warts. The insects belonging to Lepidoptera herein include not only an imago, but also stages of an embryo (egg), a larva, and a pupa.

The term “silkworm” herein means a typical silkworm (Bombyx mori), which is a kind of insects belonging to Lepidoptera, Bombycidae. The formal Japanese name is silkmoth. While silkworm is a name of the larva of the silkmoth, the silkworm generally refers to all stages of this species. The silkworm eats mulberry, and produces silk to build a cocoon of the pupa. The silkworm is also referred to as a domesticated silkworm, and is not an insect living in the wild. The ancestor of the silkworm (Bombyx mori) is considered to be Bombyx mandarina, which inhabits East Asia. Bombyx mori and Bombyx mandarina are defined scientifically as a distinct species, but a hybrid of these species has a reproductive capability. The silkworm herein includes Bombyx mandarina.

The term “silk gland” herein refers to a matched pair of organs present in the body of a mature silkworm, and means an organ that converts a large amount of proteins (amino acids) digested from mulberry leafs into two kinds of silk proteins (fibroin, sericin). The silk gland consists of a matched pair, and secretes liquid silk that becomes a source material for cocoon thread. The silk gland is divided into three parts, including a posterior silk gland, a middle silk gland, and an anterior silk gland.

The posterior silk gland refers to a thin part located at the most posterior portion, and has about 20 cm in length when elongated. This synthesizes a fibroin protein which primarily makes up cocoon thread later.

The middle silk gland refers to a sigmoidal thick part located at the middle portion, and has about 6 cm in length when elongated. This accumulates the fibroin protein by concentration of the fibroin protein transferred from the posterior silk gland, and makes the protein readily form a fiber. This also secretes the other silk protein, sericin. The sericin plays a role as adhesive that makes bundles of the fibroin proteins.

The anterior silk gland is a thin tube that has about 4 cm in length and connects to fusula, and becomes thinner as the distance to the tip decreases. A liquid fibroin protein molecule is stretched to be in a certain direction, and assembles one another to further remove water. A pair of tubes joins together into one tube at the end of the tubes, and the fibroin is ejected from fusula to become one cocoon thread.

A silkworm stops eating mulberry at the end of fifth instar (mature silkworm). The body of a mature silkworm is filled with a pair of organs (silk glands) that store a syrupy liquid (liquid silk) which is a source material of cocoon thread. The silk gland connects to fusula underneath the mouth of a silkworm via a thin thread-ejecting tube. The liquid silk is stretched to be a solid by passing through the thin thread-ejecting tube, thereby becoming cocoon thread. Furthermore, the thread that a larva ejects from the thread-ejecting tube is attached to an adjacent object, and the larva moves its head and chest like figure of “8” and pulls the thread in a series of movements, thereby pulling the cocoon thread from the silk gland one after another.

The “fibroin” herein refers to a fibrous protein accounting for about 75% of cocoon thread, and is a source material of silk. The fibroin is gloss and smooth, and has a property that water or even an alkali solution cannot dissolve it. One fibroin fiber is composed of about 2,000 fibrils, and the fibril is further composed of fine microfibrils having one hundred-thousandth mm in diameter.

The “sericin” herein refers to a protein accounting for about 25% of cocoon thread, and is easily soluble in water while playing a role of adhesive building a cocoon by making a bundle of two fibroin fibers.

The numerical value designated as % denotes a percentage (%) of the molar ratio of the sugar chain at issue to the total sugar chain.

Other terms or concepts herein are defined in detail in the Description of the Preferred Embodiments and Examples. In addition, a variety of techniques used to perform the present invention are techniques whose sources are explicitly indicated in particular, or techniques which are readily and certainly practicable for those skilled in the art on the basis of publicly-known documents, etc. For example, genetic engineering and molecular biological techniques are described in Sambrook and Maniatis, in Molecular Cloning-A Laboratory Manual, Cold Spring Harbor Laboratory Press, New York, 1989; Ausubel, F. M. et al., Current Protocols in Molecular Biology, John Wiley & Sons, New York, N.Y., 1995, and the like.

### <History of the Invention>

Neosilk Corp., Ltd. to which the present inventors belong has already developed a technology used to produce a recombinant protein in a transgenic silkworm (TG silkworm). The main features include the following three points.
(1) A host is not a cultured cell derived from a silkworm, but is an individual silkworm being an insect (or a somatic cell contained in the individual silkworm).
(2) The technique does not use a baculovirus, etc., but uses a gene-transfer technique employing transgenic technology.
(3) The expression of a desired protein is regulated by a sericin promoter or a fibroin promoter so that the protein can be expressed specifically in a silk gland or eventually in a cocoon that a silkworm builds.

Neosilk Corp. has succeeded in creating a transgenic silkworm itself secreting a recombinant protein as a component of silk thread. However, previous findings teach that a sugar chain is added to the protein produced in a silkworm, but this structure of the sugar chain is not identical to that of proteins synthesized in mammals. Because of this, this structure of the sugar chain is a problem when glycoprotein pharmaceuticals are produced in a silkworm. In addition, even if this problem is resolved by genetic engineering of a silkworm cell or silkworm body, the analysis of the structure of the sugar chain that is added in the silkworm is necessary so as to select the glycosyltransferase that is required to modify the sugar chain.

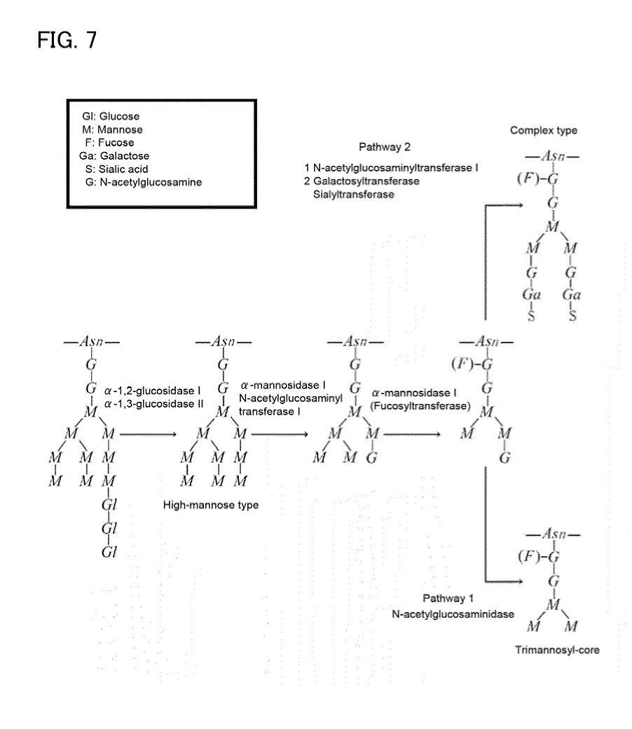

Accordingly, the present inventors have analyzed the sugar chain structure of murine IgG produced in a transgenic silkworm generated by a conventional method in the above Neosilk Corp., and have obtained the following results (FIG. 7).

(1) Obtained are a high-mannose type, and a biantennary type which is obtained by adding one or two GlcNAc to 1,3-linked mannose and 1,6-linked mannose of the trimannosyl-core structure via β-1,2-linkage and to which Gal is not linked.

(2) A low-mannose type structure, which is a distinctive feature of a sugar chain derived from an insect was not detected. In addition, α-1,3-linked fucose (hereinafter, referred to as 1,3-Fuc) that is linked to a GlcNAc residue at a reducing terminal was also not detected. The addition of α-1,6-linked fucose (hereinafter, referred to as 1,6-Fuc) is not found as well.

Here, the N-linked sugar chain of an insect including a silkworm is generally characterized by the following points.

(1) A paucimannose type (low-mannose type) is the main type, and there is no addition of galactose (hereinafter, referred to as Gal) and sialic acid.

(2) While mammals including a human have a 1,6-Fuc structure in GlcNAc binding to Asn, an insect can have a 1,3-Fuc structure in addition to the 1,6-Fuc structure.

The results obtained by performing a structural analysis of the sugar chain of murine IgG produced in a transgenic silkworm has revealed a structure completely different from the sugar chain structure of the N-linked sugar chain of the above insect, and have been a surprising finding that overturns the previous prediction.

That is, the present inventors have found that 1,3-Fuc and 1,6-Fuc is hardly detected in the sugar chain synthesized in the silk gland of a silkworm. In addition, while a previous finding teaches that the sugar chain having three or two mannoses accounts for almost all of the sugar chain synthesized in an insect, in reality, the sugar chain with five mannoses or GlcNAc is found abundant in IgG purified from a cocoon.

Specifically, according to the experiments conducted by the present inventors, it was revealed that the previously predicted results (the results showing that the sugar chain having three or two mannoses accounts for almost all of the sugar chain) were reproduced in the fat body of a silkworm, which is a tissue primarily expressing a recombinant protein in a baculovirus expression system, while, the results similar to those of an antibody (IgG) (the results showing that the sugar chain to which five mannoses or GlcNAc are linked is abundant) were obtained in the cocoon of a silkworm. Additionally, the results similar to these experimental results have been obtained by analyzing the sugar chain of the silk gland of a silkworm. That is, the experiments conducted by the present inventors have elucidated that the above sugar chain is similar to the sugar chain of CHO (Chinese hamster ovary) cells (a mammalian type, a human type) rather than that of the fat body of a silkworm.

In other words, the present inventors have found out that the sugar chain of the glycoprotein synthesized in the silk gland of an silkworm and secreted to a cocoon has a strong advantage in respect to the humanization of the sugar chain (elongation of the sugar chain, without α-1,3-fucose). This is an unexpected result completely different from the previous findings, and the present inventors further have continued the research.

After that, the present inventors have generated a transgenic silkworm expressing β-galactosyltransferase (GalT) and N-acetylglucosaminyltransferase (GnT) in the silk gland, and have analyzed the sugar chain of proteins of the cocoon. As described below, the results are yet to be improved in the ratio, but have shown a success in constructing the sugar chain in which β-Gal is linked to the terminal of GlcNAc. That is, the sugar chain of the glycoprotein produced in the silk gland of the silkworm generated in such a manner has almost achieved the humanization of the sugar chain while it still has room for improvement. As described above, according to the method for producing a glycoprotein in a silk gland of a silkworm that the present inventors have accomplished, the expression of β-galactosyltransferase, or β-galactosyltransferase and N-acetylglucosaminyltransferase together with the desired glycoprotein makes the sugar chain become humanized (elongation of the sugar chain, without α-1,3-fucose). Accordingly, the use of the silk gland as an expression tissue is considered to have tremendous merits.

### Summary of the Embodiments

Hereinafter, the embodiments of the present invention are described in detail.

According to an embodiment of the present invention, it provides a method for producing a glycoprotein having an N-glycoside-linked sugar chain in which fucose is not linked to N-acetylglucosamine at a reducing terminal of the sugar chain, the method comprising the step of expressing the glycoprotein in a silk gland cell of an insect.

According to this production method, the glycoprotein having the N-glycoside-linked sugar chain in which fucose is not linked to N-acetylglucosamine at the reducing terminal of the sugar chain can be expressed efficiently in the silk gland cell of the insect classified into Lepidoptera, and the glycoprotein expressed can be efficiently extracted. Accordingly, the glycoprotein characterized by the sugar chain structure can be produced efficiently and at low cost by using the insect cell. In addition, the glycoprotein having an N-glycoside-linked sugar chain in which fucose is not linked to N-acetylglucosamine at a reducing terminal of the sugar chain exhibits low antigenicity in vivo in mammals. Thus, it is preferred in a purpose for administering to the mammals.

### <The Case in Which the Glycoprotein is an Antibody>

In the method for producing a glycoprotein as described above, the above glycoprotein is preferred to be an antibody having a sugar chain. Since the antibody that is the above glycoprotein having the sugar chain is an antibody having an N-glycoside-linked sugar chain in which fucose is not linked to N-acetylglucosamine at a reducing terminal of the sugar chain, the superior effects which improve the ADCC activity in vivo in mammals and decrease the antigenicity are obtained.

Here, in an embodiment of the present invention, any antibodies can be used if the above antibody is a protein produced in vivo by an immune response as a result of an exogenous antigen stimulation, and has an activity of binding specifically to an antigen. Examples of the antibody include an antibody secreted from a hybridoma generated by immunizing an animal with an antigen followed by preparing a spleen cell of the immunized animal, an antibody constructed by gene recombinant technology (i.e., an antibody obtained by introducing an antibody-expressing vector, to which the antibody gene is inserted, into a host cell), and the like. Specifically, examples of the antibody include an antibody produced from a hybridoma, a humanized antibody, a human antibody, and the like.

The hybridoma means a cell producing a monoclonal antibody having a desired antigen specificity, the cell obtained by a cell fusion of both B cell obtained by immunizing a mammal except a human with an antigen and a myeloma cell derived from a mouse, etc.

Examples of the humanized antibody include a human-type chimeric antibody, a human-type complementarity determining region (hereinafter, referred to as CDR)-transplanted antibody, and the like. The human-type chimeric antibody means an antibody comprising a variable region of a heavy chain of an antibody from an animal other than a human (hereinafter, the heavy chain is referred to as an H-chain and the variable region is referred to as HV or VH) and a variable region of a light chain of an antibody from an animal other than a human (hereinafter, the light chain is referred to as an L-chain and the variable region is referred to as LV or VL) together with a constant region of a heavy chain of a human antibody (hereinafter, the constant region is referred to as a C-region or CH) and a constant region of a light chain of a human antibody (hereinafter, referred to as CL). Any of animals such as a mouse, a rat, a hamster, and a rabbit can be used as the animal other than a human if the hybridoma can be generated.

The human-type chimeric antibody can be produced by obtaining cDNAs encoding VH and VL from the hybridoma producing a monoclonal antibody, constructing an expression vector of the human-type chimeric antibody by inserting each cDNA into a host cell-expression vector having the genes encoding CH of an human antibody and CL of an human antibody, and expressing by to introducing it into the host cell. As CH of the human-type chimeric antibody, any of those belonging to human immunoglobulins (hereinafter, referred to as hIg) can be used. The hIgG class is preferred. Further, any of the subclasses including hIgG1, hIgG2, hIgG3, and hIgG4 belonging to the hIgG class can be used. In addition, as CL of the human-type chimeric antibody, any of those belonging to hIg can be used, and κ class or λ class can be used.

The human type CDR-transplanted antibody means an antibody in which the amino acid sequences of CDR of VH and VL of an antibody from an animal other than a human are transplanted into appropriate positions of VH and VL of an human antibody. The human-type CDR-transplanted antibody can be produced by constructing cDNAs encoding V-regions in which the CDR sequences of VH and VL of an antibody from an animal other than a human are transplanted into the CDR sequences of VH and VL of a human antibody, constructing an expression vector of the human-type CDR-transplanted antibody by inserting each cDNA into a host cell-expression vector having the genes encoding CH of an human antibody and CL of an human antibody, and expressing the human-type CDR-transplanted antibody by introducing the expression vector into the host cell. As CH of the human-type CDR-transplanted antibody, any of those belonging to hIg can be used. The hIgG class is preferred. Further, any of the subclasses including hIgG1, hIgG2, hIgG3, and hIgG4 belonging to the hIgG class can be used. In addition, as CL of the human-type CDR-transplanted antibody, any of those belonging to hIg can be used, and κ class or λ class can be used.

A human antibody originally means an antibody naturally present inside a human body, but includes an antibody, etc. obtained from a human antibody phage library, and a human antibody-producing transgenic animal or a human antibody-producing transgenic plant, which are generated by an advance of technologies in recent genetic engineering, cellular engineering and developmental engineering. The antibody present inside a human body can be purified from the culture in which a lymphocyte, producing the antibody, can be cultured by the isolation of the lymphocyte from human peripheral blood, the immortalization by infecting with EB virus, etc., and the cloning.

The human antibody phage library is a library in which an antibody fragment such as Fab and a single chain antibody is expressed on the surface of a phage by inserting the antibody gene prepared from human B cells into the phage gene. The phage expressing the antibody fragment having a desired antigen-binding activity can be recovered from the library by using, as an index, the activity of binding to a substrate on which an antigen is immobilized. The antibody fragment can be further converted to a human antibody molecule comprising two complete H-chains and two complete L-chains by a genetic engineering procedure.

### <Structure of the Sugar Chain>

The above mentioned sugar chains are largely divided into two kinds including a sugar chain linked to asparagine (N-glycoside-linked sugar chain) and a sugar chain linked to serine or threonine, etc., (O-glycoside-liked sugar chain). The N-glycoside-linked sugar chain described above possesses a variety of structures (Biological Chemistry Experimental Method 23-Glycoprotein Sugar Chain Research Method (Gakkai-Shuppan Center), edited by Toyoko TAKAHASHI, 1989). In any cases, it is suitable to have a common core structure that is a basis showed below. Certainly, in respect to this point, the same applies to even the case in which the glycoprotein described above is not an antibody.

That is, in the production method described above, the above glycoprotein preferably includes a glycoprotein having an N-glycoside-linked sugar chain represented by the following formula (1):

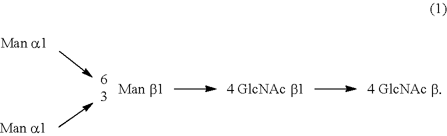

This is because in general, the sugar chain structure of a glycoprotein in an animal including a mammal and an insect to begin with is known to have such a common core structure. That is, if such the sugar chain structure is present, the antigenicity can be decreased when it is administered to the body of a mammal.

In the above structure, the terminal of the sugar chain linking to asparagine is referred to as a reducing terminal, and another terminal is referred to as a non-reducing terminal. Examples of the linkage of fucose to N-acetylglucosamine at a reducing terminal include α-1,3-linkage, α-1,6-linkage, and the like.

Examples of the N-glycoside-linked sugar chain include a high-mannose type in which only mannoses are linked to a non-reducing terminal of a core structure, a complex type which has one or a plurality of parallel branches of galactose-N-acetylglucosamine (hereinafter, referred to as Gal-GlcNAc) at a non-reducing terminal of a core structure and has a structure such as sialic acid and bisecting N-acetylglucosamine, and a hybrid type which has both branches of the high-mannose type and the complex type at a non-reducing terminal of a core structure.

In an embodiment of the present invention, the above glycoprotein preferably includes a glycoprotein having an N-glycoside-linked sugar chain comprising the sugar chain structure represented by the following chemical formulae (2), (3), and (4):

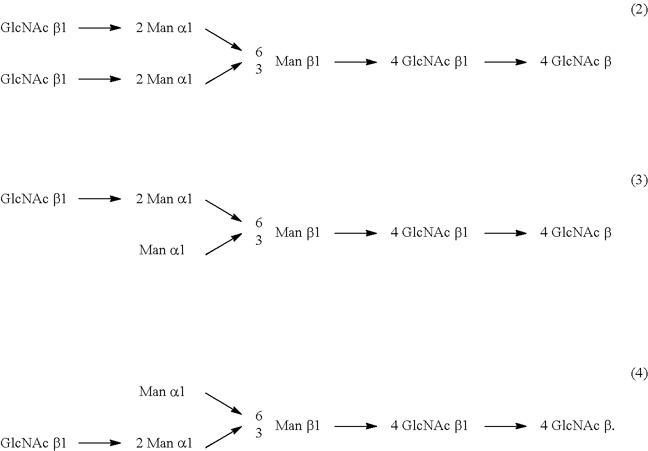

This is because in general, the sugar chain structure of a glycoprotein in a mammal is known to have such a common core structure. That is, if such the sugar chain structure is present, the antigenicity can be decreased when it is administered to the body of a mammal. In addition, this is because the stability and physiological activity of the glycoprotein can be equal to those of the glycoprotein in a mammal.

Among the glycoproteins described above, the ratio of the glycoprotein having the N-glycoside-linked sugar chain comprising the sugar chain structure represented by the chemical formulae (2), (3), and (4) is preferably 10% or more, more preferably 20% or more, and particularly preferably 30% or more.

When the ratio of the glycoprotein having the N-glycoside-linked sugar chain comprising the sugar chain structure represented by the chemical formulae (2), (3), and (4) is 10% or more, 20% or more, or 30% or more, the N-glycoside-linked sugar chain of the glycoprotein is markedly similar to the sugar chain structure of the N-glycoside-linked sugar chain of the glycoprotein produced in a mammalian cell including a human to begin with, when compared with the N-glycoside-linked sugar chain produced in a typical insect cell. It becomes what is called a “humanized sugar chain”-containing glycoprotein in a considerable extent. Since the glycoprotein is a “humanized sugar chain”-containing glycoprotein, the antigenicity can be decreased when it is administered to the body of a mammal. In addition, the stability and physiological activity of the glycoprotein can be equal to those of the glycoprotein in a mammal.

In an embodiment of the present invention, in respect to the sugar chain structure described above, the ratio of the glycoprotein having the sugar chain structure in which fucose is linked to N-acetylglucosamine at the reducing terminal of the sugar chain is preferably 5% or less, more preferably 3% or less, and most preferably 1% or less among the glycoproteins having an N-glycoside-linked sugar chain. This is because when the above glycoprotein is an antibody, the ADCC activity in vivo in a mammal improves if the ratio of the glycoprotein having the sugar chain structure to which fucose is linked in such a manner is 5% or less, 3% or less, or 1% or less. This is also because the antigenicity in vivo in a mammal can be decreased, irrespective whether the above glycoprotein is an antibody or not.

Further, among the above glycoproteins, the ratio of the glycoprotein having a sugar chain structure in which α-1,6-fucose is linked to N-acetylglucosamine at a reducing terminal of an N-glycoside-linked sugar chain is preferably 5% or less, more preferably 3% or less, most preferably 1% or less. This is preferred because when the above glycoprotein is an antibody having a sugar chain, the antibody possesses the potent ADCC activity compared with the same kind of antibodies having a typical sugar chain structure in a mammalian cell if the ratio is 5% or less, 3% or less, or 1% or less.

Here, the ADCC activity means an activity in which an antibody binding to tumor cells, etc., activates an effector cell via binding to a Fc receptor present on the surface of the effector cell (e.g., a killer cell, a natural killer cell, and an activated macrophage) through the Fc region of the antibody, and injures the tumor cells, etc. (Monoclonal Antibodies: Principles and applications, Wiley-Liss, Inc., Chapter 2.1, 1995).

Further, among the above glycoproteins, the ratio of the glycoprotein having a sugar chain structure in which α-1,3-fucose is linked to N-acetylglucosamine at a reducing terminal of an N-glycoside-linked sugar chain is preferably 5% or less, more preferably 3% or less, and most preferably 1% or less. This is because the glycoprotein possesses reduced antigenicity in vivo in a mammal compared with the same kind of glycoproteins having a typical sugar chain structure in an insect cell if the ratio is 5% or less, 3% or less, or 1% or less. Such reduced antigenicity also applies to the sugar chain structure of glycoproteins other than an antibody.

Then, the silk gland cell described above can be transformed to express β-galactosyltransferase. At this case, the above glycoprotein preferably includes a glycoprotein having an N-glycoside-linked sugar chain comprising the sugar chain structure represented by the following chemical formulae (5), (6), and (7):

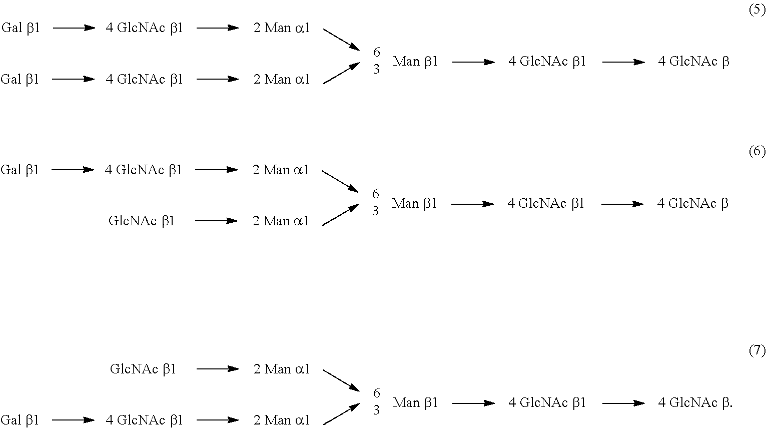

This is because in general, many of the sugar chain structures of glycoproteins in a mammal are known to have such a common core structure. That is, if such the sugar chain structure is present, the antigenicity can be decreased when it is administered to the body of a mammal. In addition, this is because the stability and physiological activity of the glycoprotein can be equal to those of the glycoprotein in a mammal.

Among the glycoproteins described above, the ratio of the glycoprotein having the N-glycoside-linked sugar chain comprising the sugar chain structure represented by the chemical formulae (5), (6), and (7) is preferably 4% or more, more preferably 7% or more, and particularly preferably 10% or more.

When the ratio of the glycoprotein having the N-glycoside-linked sugar chain comprising the sugar chain structure represented by the chemical formulae (5), (6), and (7) is 4% or more, 7% or more, or 10% or more, the N-glycoside-linked sugar chain of the glycoprotein is markedly similar to the sugar chain structure of the N-glycoside-linked sugar chain of the glycoprotein produced in a mammalian cell including a human to begin with, when compared with the N-glycoside-linked sugar chain of the glycoprotein produced in a typical insect cell. It almost completely becomes what is called a “humanized sugar chain”-containing glycoprotein. Since the glycoprotein is a “humanized sugar chain”-containing glycoprotein, the antigenicity can be decreased when it is administered to the body of a mammal. In addition, the stability and physiological activity of the glycoprotein can approach those of the glycoprotein in a mammal.

### <Method for Analyzing a Sugar Chain>

A method for analyzing a sugar chain is illustrated when the glycoprotein described above is an antibody having a sugar chain (IgG as an exemplified example). In addition, a similar method basically allows an analysis of the sugar chain even if the above glycoprotein is not an antibody having a sugar chain.

### Composition Analysis of a Neutral Sugar and an Amino Sugar

A sugar chain of IgG, as indicated above, is composed of a neutral sugar (e.g., galactose, mannose, fucose), an amino sugar (e.g., N-acetylglucosamine), and an acid sugar (e.g., sialic acid). The composition analysis of the sugar chain of an antibody can be an analysis of the composition by performing acid hydrolysis of a sugar chain by using trifluoroacetic acid, etc., and by releasing a neutral sugar or an amino sugar. Examples of the specific method include a method using a sugar chain composition analyzer (BioLC) manufactured by Dionex Corp. BioLC is an device for analyzing a sugar composition by the HPAEC-PAD (high performance anion-exchange chromatography-pulsed amperometric detection) method (Journal of Liquid Chromatography (J. Liq. Chromatogr.), 6, 1577, 1983).

The composition ratio can be analyzed by a fluorescence labeling method using 2-aminopyridine. Specifically, the sample subjected to acid hydrolysis according to a known method (Agricultural and Biological Chemistry (Agric. Biol. Chem.), 55 (1), 283-284, 1991) can be fluorescently labeled with 2-aminopyridylation, and be subjected to the HPLC analysis to calculate the composition ratio.

### Structural Analysis of a Sugar Chain

The structural analysis of the sugar chain of an antibody can be carried out by the two-dimensional sugar chain mapping method (Analytical Biochemistry (Anal. Biochem.), 171, 73, 1988; Biological Chemistry Experimental Method 23-Glycoprotein Sugar Chain Research Method (Gakkai-Shuppan Center), edited by Toyoko TAKAHASHI, 1989). In the two-dimensional sugar chain mapping method, for example, the retention time or the elution position of the sugar chain obtained by reversed-phase chromatography is plotted as an X-axis, and the retention time or the elution position of the sugar chain obtained by normal-phase chromatography is plotted as a Y-axis, respectively. Then, the structure of the sugar chain can be deduced by comparing with the results of a known sugar chain.

Specifically, after an antibody is subjected to hydrazinolysis to release a sugar chain from the antibody and the fluorescent labeling of the sugar chain by 2-aminopyridine (hereinafter, abbreviated as PA) (Journal of Biochemistry (J. Biochem.), 95, 197, 1984) is performed, the sugar chain is separated from an excessive PA reagent by gel filtration, and reversed-phase chromatography is carried out. Next, each peak of the fractioned sugar chains is subjected to normal-phase chromatography. Based on these results, the results are plotted on the two-dimensional sugar chain map, and the sugar chain structure is deduced by comparing with a spot of a sugar chain standard and a publication (Analytical Biochemistry (Anal. Biochem.), 171, 73, 1988). Further, the respective sugar chain is subjected to mass spectrometry such as MALDI-TOF-MS, the structure deduced from the two-dimensional sugar chain mapping method can be verified.

### <Method for Producing a Glycoprotein>

In an embodiment of the present invention, a glycoprotein is expressed in a silk gland cell of an insect classified as Lepidoptera. This is because the glycoprotein having an N-glycoside-linked sugar chain in which fucose is not linked to N-acetylglucosamine at a reducing terminal of a sugar chain can be surprisingly obtained contrary to conventional common technical knowledge when the glycoprotein is expressed in the silk gland cell of the insect classified as Lepidoptera.

At that occasion, the above insect is preferably a silkworm. This is because in the case of the silkworm, the feeding procedure is established in sericultural industry, and the above glycoprotein can be yielded efficiently in a large quantity due to a superior ability of ejecting a cocoon from a silk gland.

More specifically, Neosilk Corp. to which the present inventors belong keeps an eye on an ability of a silkworm for synthesizing a silk protein, and has developed a transformed silkworm that secretes a large amount of a recombinant protein together with a silk protein into a cocoon. Then, the silkworm produces 0.3 to 0.5 g of a cocoon per insect. However, almost all of them are a silk protein such as fibroin synthesized in a posterior silk gland and sericin synthesized in a middle silk gland. As described above, a silkworm is an organism having a superior protein synthesis ability, and the use of this ability allows the production of a recombinant protein such as an antibody medicine economically and in a large amount.

Furthermore, the above silk gland cell is preferably a middle silk gland cell. This is because a recombinant protein can be efficiently expressed in the middle silk gland due to localization of the recombinant protein in a sericin layer relatively soluble in water and due to the presence in the vicinity of fibroin when the above glycoprotein is expressed in the sericin portion, since sericin is synthesized in the middle silk gland.

That is, in the method for producing a glycoprotein according to an embodiment of the present invention, the step of expressing the above glycoprotein preferably comprises a step of producing a cocoon containing the above glycoprotein in the silk gland of the above insect body that is genetically modified to express the above glycoprotein. Here, as it is to be noted so as not to cause misunderstanding, of note is that producing a cocoon containing the above glycoprotein in the silk gland of the insect body inevitably means expressing the above glycoprotein in the silk gland cell of the insect.

A step of extracting the above glycoprotein preferably comprises the steps of ejecting a cocoon containing the above glycoprotein from the above silk gland, and extracting the above glycoprotein from the above ejected cocoon. This is because the recombinant protein can be efficiently expressed in the silk gland and the recombinant protein contained in the cocoon can be extracted due to localization of the recombinant protein in a fibroin layer or a sericin layer when the above glycoprotein is expressed in the fibroin or sericin portion, since fibroin or sericin is synthesized in the silk gland.

At that occasion, when the above glycoprotein is an antibody having a sugar chain, the step of expressing the above glycoprotein preferably comprises a step of using the above insect body that is modified to coexpress a heavy chain and a light chain of an antibody constituting the foregoing antibody in the above insect body. As such a configuration is taken, a transgenic silkworm that secretes a functional antibody molecule (a heterodimer) in which structural genes of an H-chain and L-chain of the antibody are incorporated in a chromosome, in which two H-chain molecules and two L-chain molecules are linked via a disulfide bond, and which has an activity of binding to an antigen can be used. Accordingly, the silkworm secretes a heterodimer that is a complete antibody molecule primarily into a cocoon, and there is no case of uncontrollably secreting an incomplete molecule such as an H-chain dimer that can be seen in other insect cells. As a result, the cocoon does not contain an incomplete molecule such as an H-chain dimer. Because of this, the heterodimer that is a complete antibody molecule can be readily collected from the cocoon.

In the above insect body, the step of expressing the above glycoprotein preferably comprises a step of using an insect body in which both a gene encoding the above antibody heavy chain and a gene encoding the above antibody light chain are placed expressably downstream of a sericin promoter in the genome of the above insect, and a step of generating a cocoon containing the above antibody in the sericin portion as the above cocoon. In this case, the step of extracting the above glycoprotein preferably comprises a step of extracting the above antibody from the above sericin portion of the above cocoon into an extracting solution, following immersing the above cocoon into the extracting solution.

This is because silk thread constituting a cocoon have a structure in which a sericin layer is present in the vicinity of a fibroin layer located at the center. The silk gland synthesizing a silk protein is divided functionally and morphologically into a posterior silk gland, a middle silk gland, and an anterior silk gland. Sericin constituting a sericin layer is synthesized in the middle silk gland, and fibroin constituting a fibroin layer is synthesized in the posterior silk gland. If a tissue expressing an antibody is the middle silk gland, the antibody is secreted into a sericin layer of the silk thread. In contrast, if a tissue expressing an antibody is the posterior silk gland, the antibody is secreted into a fibroin layer of the silk thread.

In order to generate a transgenic silkworm secreting an antibody into a sericin layer, a promoter that induces a gene expression in the middle silk gland cell can be placed, for example, upstream of the respective structural gene in addition to the structural genes of the antibody H-chain and L-chain in the vector so as to express the antibody H-chain and L-chain in the middle silk gland. Examples of the promoter that induces a gene expression in the middle silk gland can include, for example, a promoter of sericin 1 gene or sericin 2 gene.

Use of such an expression vector allows a transgenic silkworm secreting an antibody into a sericin layer to be produced by a single gene-introducing operation. On the other hand, a transgenic silkworm secreting an antibody into a sericin layer can also be generated by a plurality of gene-introducing operations. For example, a silkworm expressing an antibody H-chain in a middle silk gland and a silkworm expressing an antibody L-chain in a middle silk gland are each generated separately by using the above expression vector set. Then, they are crossed to select a silkworm having both the antibody H-chain and L-chain from the next generation, and the silkworm secreting an antibody into a sericin layer may be generated.

Alternatively, a transgenic silkworm secreting an antibody into a sericin layer may be generated by introducing an expression vector expressing the antibody L-chain into a silkworm expressing the antibody H-chain or by introducing an expression vector expressing the antibody H-chain into a silkworm expressing the antibody L-chain.

Of course, there is no intention to exclude a method for generating a transgenic silkworm secreting an antibody into a fibroin layer. A transgenic silkworm secreting an antibody into a fibroin layer can be generated by using a vector integrating a promoter that induces a gene expression in the posterior silk gland cell upstream of the respective structural gene in addition to the structural genes of the antibody H-chain and L-chain so as to express the antibody H-chain and L-chain in the posterior silk gland. Examples of the promoter that induces a gene expression in the posterior silk gland cell can include, for example, a promoter of fibroin L-chain gene, fibroin H-chain gene, fibrohexamerin gene, or the like.

A transgenic silkworm may be generated by a plurality of gene-introducing operations by using the above expression vector set in the case of generating a transgenic silkworm secreting an antibody into a fibroin layer as well as in the case of generating a transgenic silkworm secreting an antibody into a sericin layer. A silkworm expressing an antibody H-chain in a posterior silk gland and a silkworm expressing an antibody L-chain in a posterior silk gland each are generated separately. Then, they are crossed to select a silkworm having both the antibody H-chain and L-chain from the next generation, and the silkworm secreting an antibody into a fibroin layer may be generated. Alternatively, the transgenic silkworm secreting an antibody into a fibroin layer can be generated by introducing an expression vector expressing the antibody L-chain into a silkworm expressing the antibody H-chain or by introducing an expression vector expressing the antibody H-chain into a silkworm expressing the antibody L-chain.

These expression vectors have a function to introduce a gene into a chromosome of a silkworm. For example, if a partial sequence of a DNA-type transposon derived from an insect is integrated, it is possible to introduce a gene into a chromosome of a silkworm. Specifically, the vector is a plasmid vector having a pair of inverted repeat sequences present at the ends of the DNA-type transposon, and is an expression vector in which a gene sequence (i.e., an antibody gene and a promoter) that will be inserted in a chromosome is integrated in the region between the pair of inverted repeat sequences. As an insect-derived DNA-type transposon, piggyBac, mariner (Insect Mol. Biol. 9, 145-155, 2000), and Minos (Insect Mol. Biol. 9, 277-281, 2000) have been known. However, among them, the sequence derived from piggyBac has been used most frequently. In order to produce a transgenic silkworm, this plasmid, together with piggyBac's transposase expression vector (helper plasmid), is microinjected into silkworm eggs. This helper plasmid is a recombinant plasmid vector in which one or both of the inverted repeat sequences of piggyBac are absent, and only piggyBac's transposase gene region is substantially integrated. In the helper plasmid, for a promoter to express the transposase, the endogenous transposase promoter itself may be used, or the actin promoter of a silkworm, HSP promoter of Drosophila, and the like can be used. In order to facilitate the screening of the silkworms of the next generation, a marker gene can be simultaneously integrated into the vector in which a polynucleotide that will be inserted is integrated.

The larvae (F0 generation) hatched from silkworm eggs microinjected with the vector are raised. All of the resulting silkworms of F0 generation are crossed with a wild-type silkworm, or the silkworms of the F0 generation are crossed among them. Then, a transgenic silkworm is selected from the silkworms of the next generation (F1 generation). The selection of the transgenic silkworm is performed by, e.g., a PCR method or Southern blotting method. In addition, when a marker gene is integrated, the selection can be possible to use the phenotype. For example, when a fluorescent protein gene such as GFP is used as a marker gene, the selection can be performed by detecting fluorescence emitted from the fluorescent protein by irradiating silkworm eggs or larvae of F1 generation with an excitation light. According to the method described above, the transgenic silkworm can be generated.

For the recovery of a functional antibody molecule from a cocoon of the transgenic silkworm, the antibody is collected from a sericin layer when the antibody is localized in the sericin layer, and the antibody is collected from a fibroin layer when the antibody is localized in the fibroin layer. The recovery of the antibody from the sericin layer is particularly readily carried out. Since sericin constituting a sericin layer is hydrophilic, the recombinant protein localized in this layer can be extracted without using a solution that can denature a protein. The extracting solution for extracting an antibody from the sericin layer has no particular limitation if the extraction of the antibody is possible. For example, the extracting solution may be a solution comprising neutral salts, a detergent, or a solution containing a reagent, etc., for performing the extraction efficiently as well. In order to extract an antibody from a cocoon by using these extracting solutions, for example, a method for immersing a fragmented cocoon into the extracting solution and stirring it can be used. In addition, the cocoon may be subjected to a micropowdering process before extraction, or may be subjected to a mechanical process such as sonication in conjunction with the foregoing process at the extraction.

In addition, a silk gland cell of the above silkworm may be transformed to express β-galactosyltransferase. If the above silk gland cell is transformed to express β-galactosyltransferase, β-galactosyltransferase is similarly expressed in an endogenous silk gland of a silkworm body, a silk gland tissue extirpated from the silkworm body, or a silkworm gland cell obtained from the silk gland tissue extirpated from the silkworm body. Then, the glycoprotein having the N-glycoside-linked sugar chain comprising the sugar chain structure represented by the above formulae (5), (6), and (7) can be produced by further adding β-galactose to the non-reducing terminal of the N-glycoside-linked sugar chain due to the function of this enzyme.

Then, the N-glycoside-linked sugar chain of the glycoprotein is markedly similar to the sugar chain structure of the N-glycoside-linked sugar chain of the glycoprotein produced in a mammalian cell including a human to begin with when compared with the N-glycoside-linked sugar chain of the glycoprotein produced by a typical insect cell. It almost completely becomes what is called a “humanized sugar chain”-containing glycoprotein. Since the glycoprotein is a “humanized sugar chain”-containing glycoprotein, the antigenicity can be decreased when it is administered to the body of a mammal. In addition, the stability and physiological activity of the glycoprotein can be equal to those of the glycoprotein in a mammal.

In addition, the silk gland cell described above can be transformed to express N-acetylglucosaminyltransferase. If this is accomplished, the production of the glycoprotein having the N-glycoside-linked sugar chain comprising the sugar chain structure represented by the above formulae (2), (3), and (4) can be increased by further adding N-acetylglucosamine to the non-reducing terminal of the N-glycoside-linked sugar chain. As a result, the production of the glycoprotein having the N-glycoside-linked sugar chain comprising the sugar chain structure represented by the above formulae (5), (6), or (7) can be further increased by further adding β-galactose to the non-reducing terminal of the sugar chain structure represented by the above formulae (2), (3), or (4) due to the function of β-galactosyltransferase expressed together with N-acetylglucosaminyltransferase.

As described above, the embodiments of the present invention are illustrated. However, they are an example of the present invention, and a variety of configurations other than the above can be adopted.

Besides, although the phrase “expressing a glycoprotein in a silk gland cell of an insect” in the above embodiment is used, of note is that the phrase includes an intention including the expression in a cellular level, the expression in a tissue level, and the expression in an organism level.

As described just in case, for example, when the expression in a cellular level is performed, the silk gland cells may be cultured in a bioreactor. When the expression in a tissue level is performed, the silk gland tissue may be cultured. On the other hand, when the expression in an organism level is performed, it may be possible to express the glycoprotein in a silk gland of an insect body classified into Lepidoptera and to eject outside the organism by including the glycoprotein in a cocoon. If they are performed, the technology available in sericultural industry can be applied as it is, and there is an advantage that an equipment investment such as a bioreactor is unnecessary when compared with the case of production in a cellular level.

Additionally, the above embodiments do not specify a particular strain of silkworms, and any of the silkworms can be used. However, use of the cell of the silkworm from the commercial variety of strains such as pnd-w1 strain and Kinshu is particularly preferred among various kinds of silkworms. This is because when the cell of the silkworm from the commercial variety of strains such as pnd-w1 strain and Kinshu is used, the ratio of the glycoprotein having the sugar chain structure in which fucose is linked to N-acetylglucosamine at the reducing terminal of the sugar chain is demonstrated to be 5% or less among the proteins having the N-glycoside-linked sugar chain by the experimental data in Examples described below.

### EXAMPLES

Hereinafter, although the present invention is illustrated by Examples, the present invention is not limited to these Examples.

### Example 1

cDNA Cloning of an Antibody H-Chain and an Antibody L-Chain

mRNA was extracted from a murine hybridoma producing IgG antibody in which human IgG is an antigen, and cDNA was synthesized by using reverse transcriptase and oligo (dT) primers. cDNA encoding each ORF of the antibody H-chain and the antibody L-chain was obtained by PCR using the synthesized cDNA as a template.

The primer sequences used to amplify the cDNA of the antibody H-chain were 5′-GATATCCACCATGGCTTGGGTGTGGAC-3′ (SEQ ID NO: 1) and 5′-GATATCTTATCATTTACCAGGAGAGTGGGA-3′ (SEQ ID NO: 2), and the primer sequences used to amplify the cDNA of the antibody L-chain were 5′-GATATCCACCATGGTGTCCACTTCTCAGCTC-3′ (SEQ ID NO: 3) and 5′-GATATCTTACTAACACTCATTCCTGTTGAAGCT-3′ (SEQ ID NO: 4).

The site recognition sequence of EcoRV restriction enzyme was attached to each of the 5′-ends of the primers. The resulting PCR product was cloned into pCR4blunt-TOPO (Invitrogen). The nucleotide sequence was analyzed by a DNA sequencer, and the PCR product was verified to be the cDNAs encoding the antibody H-chain and the antibody-L-chain. The vectors containing the antibody H-chain and the antibody L-chain cDNAs are designated hereinafter as IgG(H)/pCR4 and IgG(L)/pCR4, respectively.

### Example 2

### Construction of a Vector for Producing a Transgenic Silkworm

The double-stranded oligonucleotide annealing 5′-end phosphorylated oligonucleotides 5′-AATTCCTTAAGCTCGAGTCGCGA-3′ (SEQ ID NO: 5) and 5′-AATTTCGCGACTCGAGCTTAAGG-3′ (SEQ ID NO: 6) was prepared.

This double-stranded oligonucleotide had restriction enzyme recognition sequences of AflII, XhoI, and NruI, and have a structure in which both the ends can ligate into EcoR1 site. This double-stranded oligonucleotide was inserted into the EcoR1 site of pBac [3xP3-DsRed/pA] (Nat. Biotechnol. 21, 52-56, 2003) that is a piggyBac vector having a red fluorescent protein (DsRed) gene expressing in the eyes and the nervous system as a marker gene so as to insert the restriction enzyme recognition sequences of AflII, XhoI, and NruI into pBac [3xP3-DsRed/pA] vector.

Further, Example 1 of JP-A-2006-109772 discloses “a vector having a firefly luciferase gene downstream of hr3 and sericin 1 promoter”. By PCR using this vector as a template, the DNA fragment that contained the hr3 and sericin 1 promoter and had XhoI sites at both the ends was amplified. The primers used were 5′-CTCGAGGATATCGAATTCCTGCAGCC-3′ (SEQ ID NO: 7) and 5′-CTCGAGCCCGATGATAAGACGACTATG-3′ (SEQ ID NO: 8).

After the amplified DNA fragment was digested with XhoI, it was inserted into the XhoI site of pBac [3xP3-DsRed/pA] in which the AflII; XhoI, and NruI restriction enzyme recognition sequences were inserted. This was designated as pMSG1.1R. The vector in which DsRed gene as a marker gene of the above vector was replaced with a green fluorescent protein (Monster GreenGFP) (hMGFP) was constructed. This was designated as pMSG1.1MG.

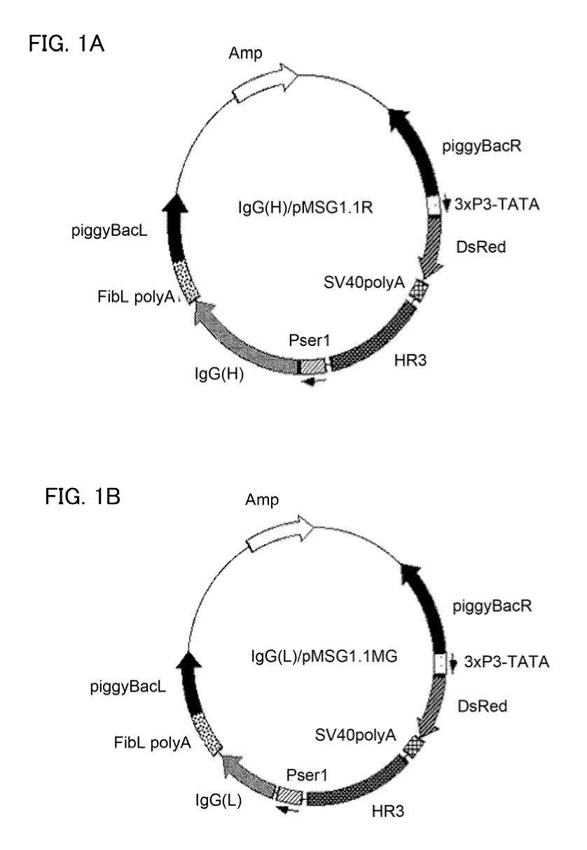

The cDNA fragments of the antibody H-chain and L-chain was cleaved with EcoRV in IgG(H)/pCR4 and IgG(L)/pCR4 obtained in Example 1, and the fragments were inserted into the above pMSG1.1R and pMSG1.1MG, respectively. The vector expressing the antibody H-chain or the antibody L-chain was constructed as IgG(H)/pMSG1.1R or IgG(L)/pMSG1.1MG (FIG. 1). Besides, in FIG. 1, the meanings of the respective abbreviations were as follows:
piggyBacR: 3′-end sequence of piggyBac;
3xP3-TATA: a promoter inducing expression in the eyes and the nervous system;
DsRed: a red fluorescent protein gene;
hMGFP: a green fluorescent protein (Monster GreenGFP) gene;
SV40 polyA: a SV40-derived poly(A) addition signal;
Pser1: a silkworm sericin 1 gene promoter;
HR3: BmNP hr3;
IgG (H): an H-chain gene of a murine IgG antibody;
IgG (L): an L-chain gene of a murine IgG antibody;
FibL polyA; a silkworm fibroin L-chain poly(A) addition signal;
piggyBacL: 5′-end sequence of piggyBac; and
Amp: an ampicillin antibiotic resistance gene

### Example 3

Generation of a Transgenic Silkworm Secreting an Antibody H-Chain and an Antibody L-Chain into a Sericin Layer

After IgG(H)/pMSG1.1R was purified by cesium chloride ultracentrifugation, each was mixed with a helper plasmid, pHA3PIG (Nat. Biotechnol. 18, 81-84, 2000), to achieve the ratio of 1:1 by the plasmid amount. After further ethanol precipitation, IgG(H)/pMSG1.1R and pHA3PIG were dissolved in an injection buffer (0.5 mM phosphate buffer pH 7.0, 5 mM KCl) so that each concentration became 200 μg/ml. This DNA solution containing IgG(H)/pMSG1.1R was microinjected with a volume of about 15 to 20 nl per egg into preblastoderm-stage silkworm eggs (silkworm embryos) 2 to 8 hours after spawning. Total 2,854 eggs were microinjected. The above similar operation was performed on IgG(L)/pMSG1.1MG, and total 3,154 eggs were microinjected.

The eggs microinjected with these vector DNAs (IgG(H)/pMSG1.1R and IgG(L)/pMSG1.1MG) were incubated at 25° C., and 516 and 678 eggs, respectively, were hatched. Newly hatched silkworm continued to be raised, and the resulting reproductive imagos were crossed to yield 138 and 132 groups, respectively, of F1 egg masses. The F1 egg masses at Day 5 to 6 after the spawning date were observed with a fluorescent stereomicroscope, and eggs of a transgenic silkworm that emits red or green fluorescence from the eyes and the nervous system were screened. As a result, the egg masses containing the eggs of the transgenic silkworm (11 groups and 15 groups, respectively) were obtained. The resulting egg masses were subjected to hatching, and were raised. The transgenic silkworms derived from nine groups and eight groups, respectively, of the egg masses built a normal cocoon, became a pupa, and further emerged from a pupa to become a reproductive imago. Then, they were crossed with a wild-type silkworm to establish a transgenic strain.

### Example 4

### Generation of a Transgenic Silkworm Coexpressing an Antibody H-Chain and an L-Chain

Transgenic silkworm imagos having the cDNA encoding the antibody H-chain or the antibody L-chain obtained in Example 3 in the genome were crossed to generate a silkworm coexpressing the H-chain and the L-chain. Any one of the strains out of two kinds of silkworms obtained in Example 3 was crossed each other, and the eggs laid were raised. At the fourth instar stage, the fluorescent color of the marker gene expressed in the eyes (H-chain: red fluorescence of DsRed, L chain: green fluorescence of hMGFP) was observed, and the silkworms expressing both the H-chain and the L-chain (emitting yellow fluorescence due to mixing of red and green fluorescence) were selected.

The resulting silkworms expressing the H-chain and the L-chain were crossed with “a transgenic silkworm integrating the polynucleotide expressing baculovirus IE1”. From the silkworms of the next generation, a transgenic silkworm expressing three genes including an H-chain, an L-chain, and IE1 was selected. This silkworm was raised at 25° C., and was made to build a cocoon.

### Example 5

### Extraction and Purification of an Antibody

After 1 g of cocoons were ground with a homogenizer, they were suspended in 100 ml of 3 M urea and 50 mM Tris-HCl buffer, pH 8.0, and were incubated at 4° C. for 24 hours. The cocoon fragments were removed by centrifugation, and the extracting solution containing an antibody was collected. This extracting solution was dialyzed against 20 mM phosphate buffer, pH 7.0, and was then applied on a protein G column (GE Healthcare, Inc.). The column was washed with 20 mM phosphate buffer, pH 7.0, and the absorbed antibody was eluted with 0.1 M glycine hydrochloride, pH 2.7. Finally, 1 M Tris was added to the extracting solution to neutralize it. From the above operation, 2.4 mg of the purified antibody was obtained.

### Example 6

### Structural Analysis of the Sugar Chain Added to an Antibody

The inventors cleaved and fluorescently labeled the sugar chain of murine IgG (1.2 mg) expressed in a silkworm in accordance with a standard method including the following protocol so as to subject to pyridylamination of the sugar chain added to the murine IgG expressed in the silkworm.

The experimental protocol illustrating the preparation of the pyridylaminated sugar chain of the murine IgG expressed in a silkworm was as follows:
Mouse IgG;
   Lyophilization;
   Hydrazinolysis (100° C., 10 h);
   N-Acetylation;
   Pyridylamination;
   Phenol/Chloroform Extraction;
   RP-cartridge; and
PA-oligosaccharides.

In addition, while hydrazinolysis was used to cleave the sugar chain from a glycoprotein at this occasion, this method is different from the cleavage using an enzyme, and has advantages that the sugar chain can be cleaved regardless of the structure of the sugar chain and the protein, and that the cleavage reaction is quantitative.

Additionally, the pyridylamination method used in the fluorescent labeling of the sugar chain has advantages as follows: it is possible to introduce a pyridylamino group into any reducing terminals of the sugar chain;
the reaction is quantitative;
it is possible for a pyridylaminated sugar chain (PA sugar chain) to be detected with extremely high sensitivity;
it is superior in a quantitative capability;
the separation by reversed-phase HPLC is excellent;
the pyridylamino group does not detach during chemical reactions such as acid hydrolysis, Smith degradation, methanolysis, and acetolysis in which the pyridylamino group is used in the sugar chain structural analysis; and
the method has an excellent match with an analyzer such as a mass spectrometer.

Specifically, 1.2 mg of the purified antibody was lyophilized, and anhydrous hydrazine was added to treat it at 100° C. for 10 hours. Then, in accordance with standard methods, the reducing terminal of the sugar chain cleaved by hydrazinolysis was subjected to N-acetylation, and the reducing terminal was further pyridylaminated (PA).

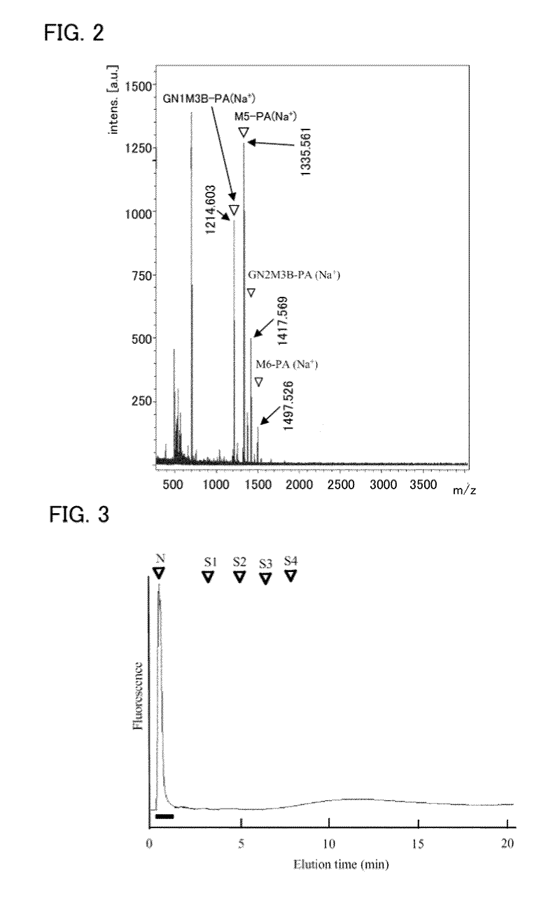

Next, in order to perform mass spectrometry of the sugar chain of the murine IgG expressed in a silkworm, the PA sugar chain as obtained above was analyzed with a mass spectrometer (FIG. 2). As a result, M5 and M6 that are a high-mannose type sugar chain were detected as shown in FIG. 2. In addition, the sugar chain that did not have galactose at the non-reducing terminal was also detected.

Then, in order to perform the sugar chain structural analysis of the sugar chain of the murine IgG expressed in a silkworm, the PA sugar chain as obtained above was further subjected to the structural analysis as indicated in the following procedure.

The experimental protocol illustrating the procedure of the structural analysis of the pyridylaminated sugar chain as obtained above was as follows:
PA-oligosaccharides.
   Mono-Q HPLC
   Size-fractionation HPLC
   Reversed-phase HPLC
   MALDI-TOF-MS

Specifically, the PA sugar chain prepared from the murine IgG expressed in a silkworm was separated by Mono-Q HPLC. FIG. 3 showed the results obtained by separating the resulting PA sugar chain by anion-exchange HPLC (Mono Q HPLC). Arrows in FIG. 3 denoted elution positions including those of a neutral sugar (N), a sugar chain to which one sialic acid was linked (S1), a sugar chain to which two sialic acids were linked (S2), a sugar chain to which three sialic acids were linked (S3), and a sugar chain to which four sialic acids were linked (S4). Since a peak was detected in the only neutral sugar portion, the portion was collected and subjected to the next experiment.

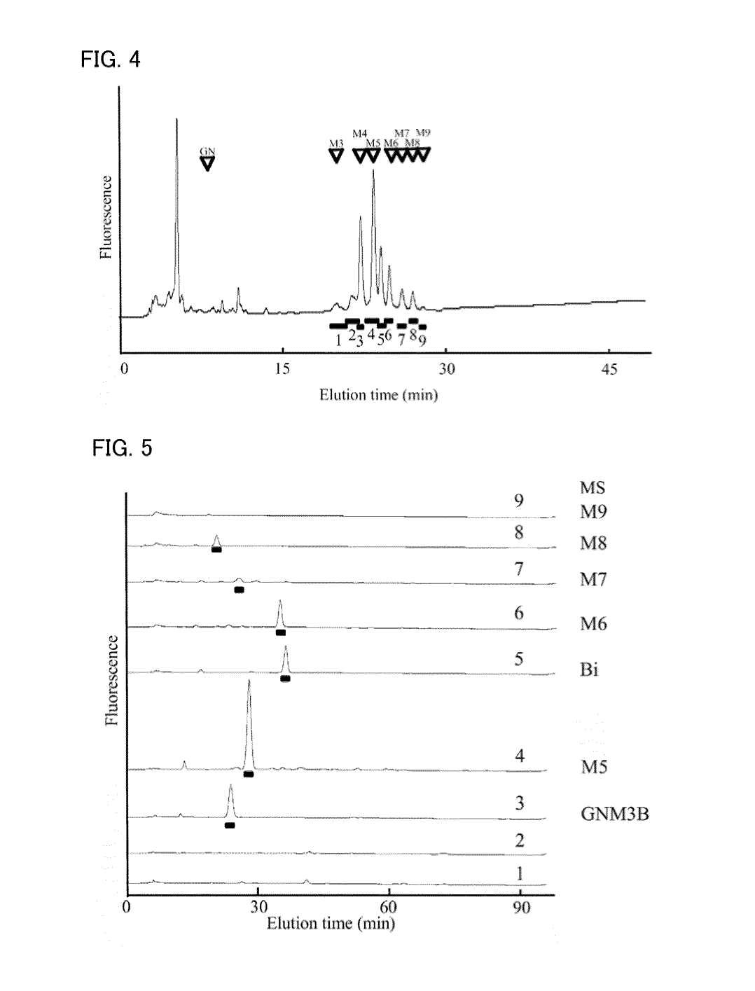

Next, the PA sugar chain prepared from the murine IgG expressed in a silkworm was separated by size-fractionation HPLC. FIG. 4 showed the results obtained by separating the resulting neutral sugar fraction by size-fractionation HPLC. For the arrows in FIG. 4, GN demoted the elution position of N-acetylglucosamine-PA, and M denoted the elution positions of the high-mannose sugar chain. As a result of the analysis, nine peaks were obtained, and each was collected, concentrated, and subjected to the next experiment by using each of them.

Next, the PA sugar chain prepared from the murine IgG expressed in a silkworm was separated by reversed-phase HPLC. FIG. 5 showed the results obtained by separating the nine fractions obtained by size-fractionation HPLC by reversed-phase HPLC. The right side of FIG. 5 showed the results obtained/by analyzing each fraction by mass spectrometry. Since six major peaks were detected, each was collected, and subjected to the two-dimensional sugar chain mapping and the structural analysis using a mass spectrometer.

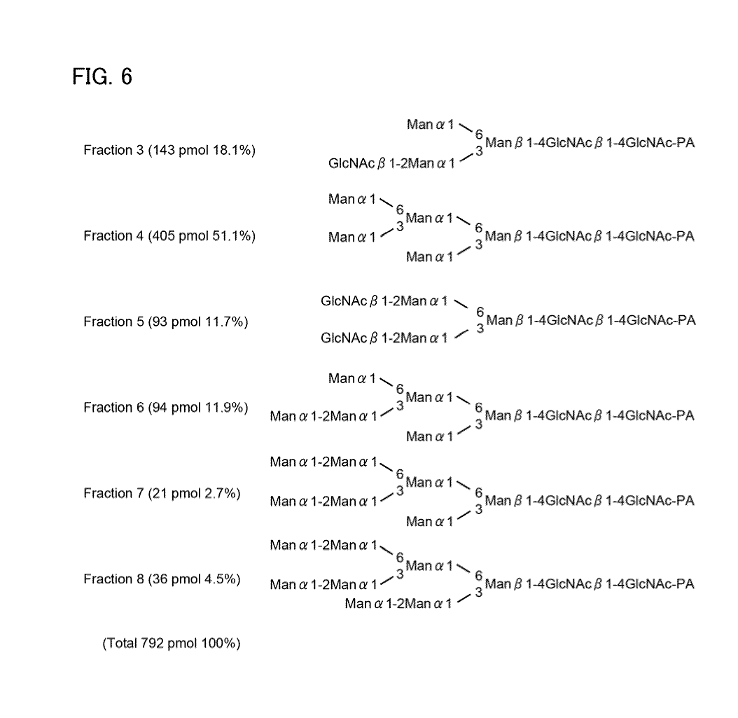

FIG. 6 showed the results of a summary of the sugar chain structures determined at this time as the structures of the PA sugar chain prepared from the murine IgG expressed in a silkworm. The “Fraction No.” in FIG. 6 represented the fraction corresponding to that in FIG. 5. The numerical values inside the parenthesis in the figure denoted the number of moles of the sugar chain contained in Fraction No. 3 to No. 8, and the percentage (%) of the number of moles of each fraction to the total number of moles (792 pmol). Then, 1.2 mg (8,000 pmol) of the murine IgG expressed in a silkworm was estimated to contain 16,000 pmol of the sugar chain. The total number of moles of the sugar chain contained in Fraction No. 3 to No. 8 was 792 pmol, and the recovery rate of the sugar chain was 5%.

As shown in FIG. 6, a sugar chain was linked to the antibody (murine IgG) that was expressed in a middle silk gland in a silkworm and that was secreted into a sericin layer of cocoon thread. There are mainly six kinds of the sugar chain structure. For any of these six kinds of the sugar chain, there existed no presence of fucose (α-1,6-fucose) that was linked to position 6 of GlcNAc present at the basal portion of the sugar chain and fucose (α-1,3-fucose) that was linked to position 3 of GlcNAc. This revealed that almost all of the sugar chains linked to an antibody expressed in a middle silk gland of a silkworm are not subjected to fucosylation. Since the ratio of the least amount of the sugar chain was 2.7% among the sugar chains detected, the ratio of the fucosylated sugar chain was estimated to be 5% or less.

In addition, the paucimannose type (low-mannose type) that was said to be characteristic of the sugar chain derived from an insect in the previous findings was not detected. Alternatively, many of the sugar chains having the structure of the high-mannose type and the biantennary type in which one or two GlcNAc were linked via β-1,2-linkage to 1,3-linked mannose and 1,6-linked mannose having the trimannosyl-core structure (paucimannose), and in which Gal was not linked thereto were detected.

### Example 7

Sugar Chain Structural Analysis of a Silkworm Tissue (a Middle Silk Gland, a Fat Body) and a Cocoon from pnd-w1 Strain and a Strain of the Commercial Variety (Kinshu)

The following three reasons were conceived as a reason for the results obtained in Example 6:
A) due to the variety of the silkworm;
B) due to the tissue of the silkworm; and
C) due to the protein expressed.

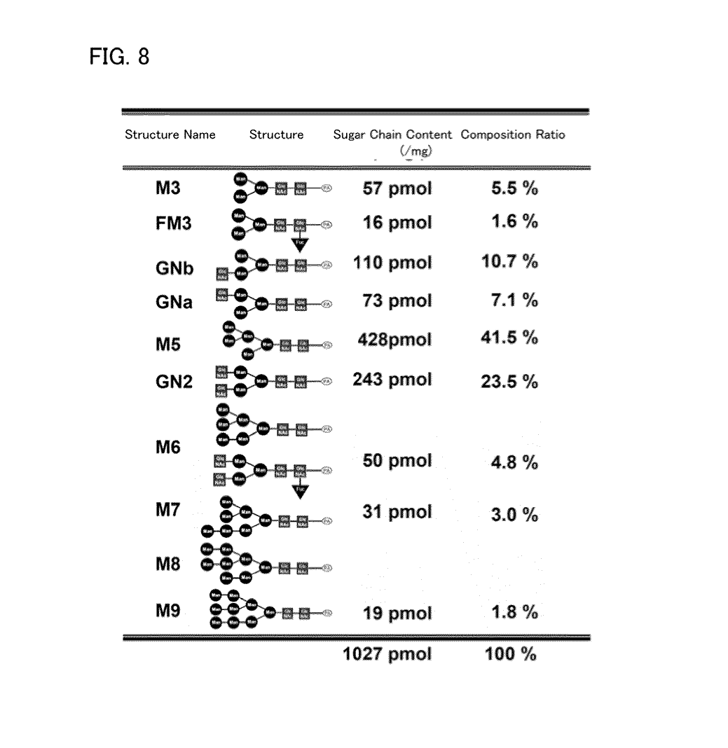

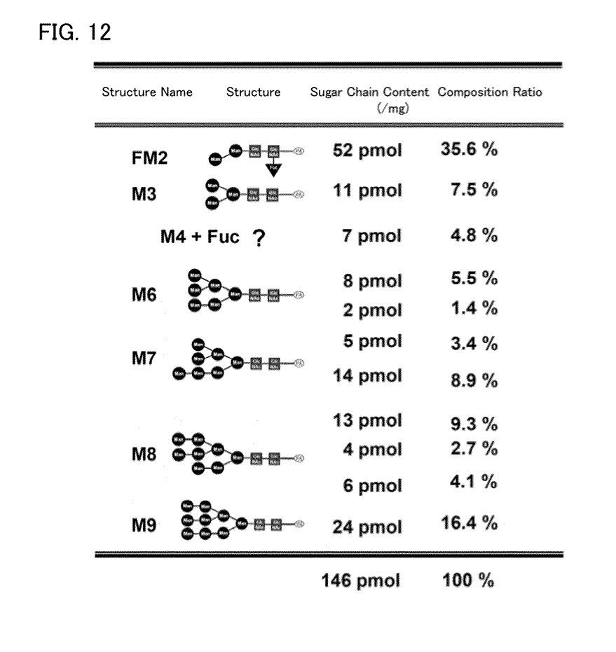

In order to investigate them, the sugar chain structural analysis was performed in accordance with the foregoing method in respect to two kinds of tissues (e.g., a middle silk gland and a fat body) and a cocoon from two kinds of silkworm strains including pnd-w1 strain and a strain of the commercial variety (Kinshu). FIG. 8 to FIG. 13 showed the results obtained by the sugar chain structural analysis. Besides, the numerical values designated as % in FIG. 8 to FIG. 13 denoted the percentage (%) of the presence ratio calculated from the total number of moles of the major peaks collected by reversed-phase HPLC. In addition, the structural abbreviations such as structural name M6, M7, M8, and M9, which were a high-mannose type, included a structural isomer.

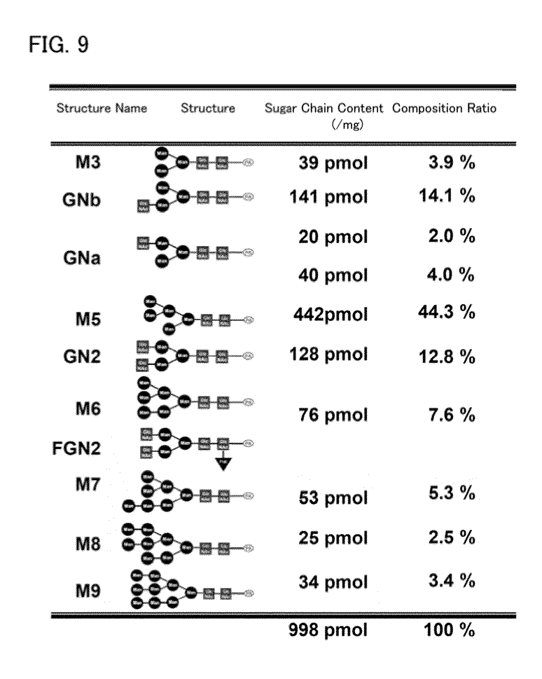

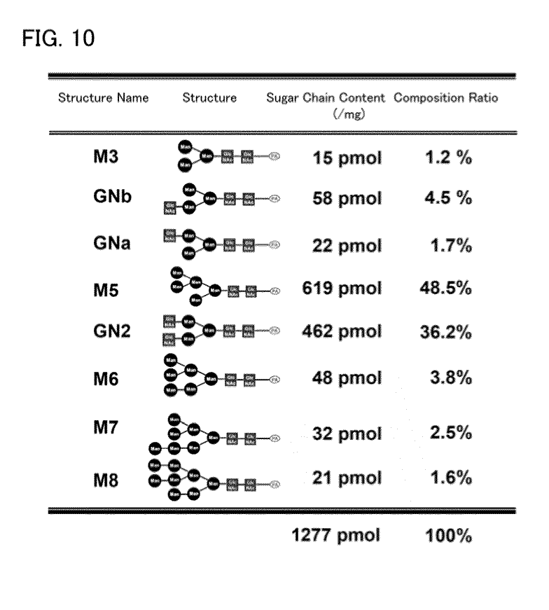

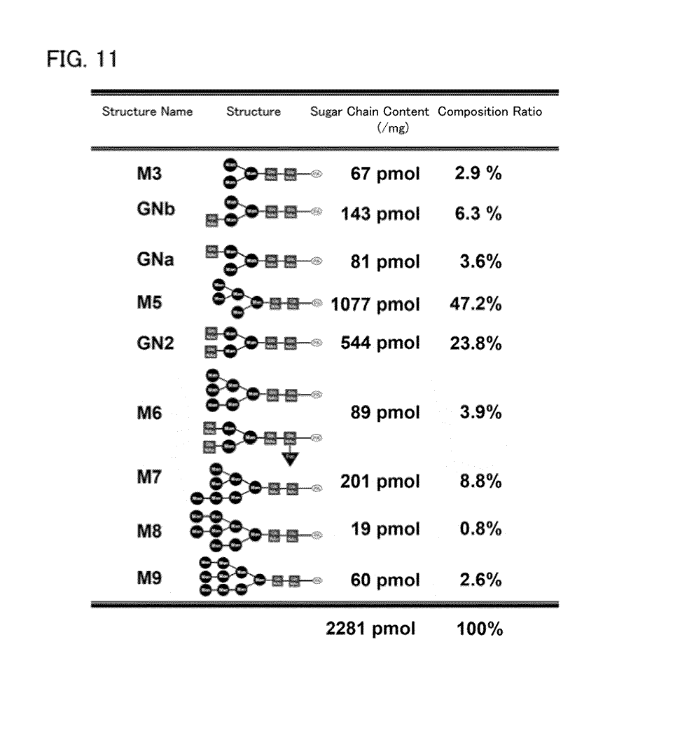

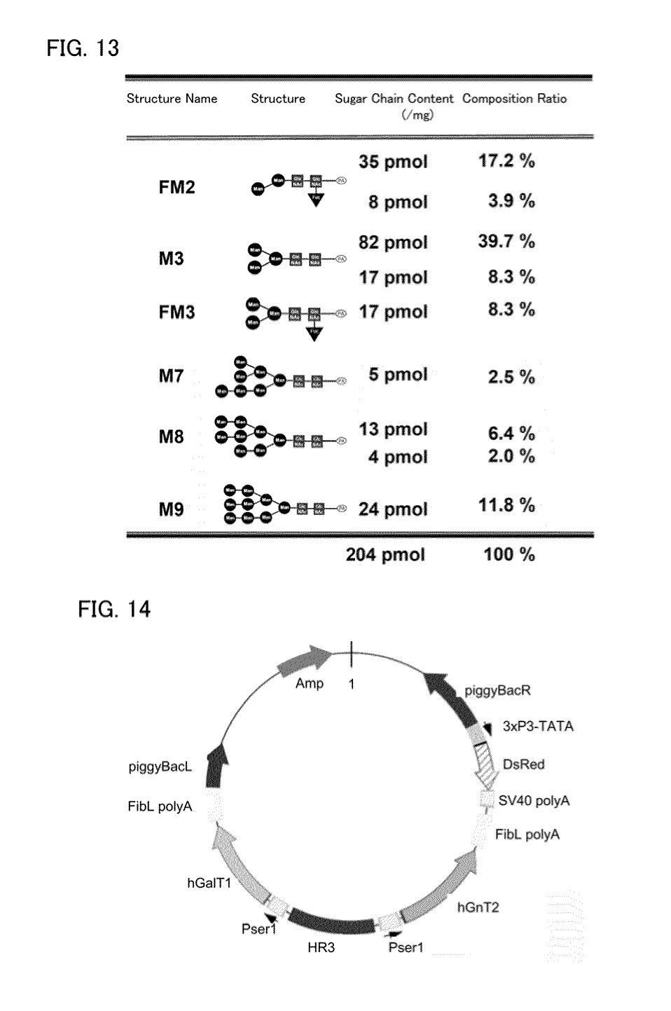

The sugar chain obtained from the middle silk gland of the pnd-w1 strain had a structure similar to the sugar chain of the murine IgG purified from the cocoon. That is, a paucimannose type that was considered to be most abundant among the sugar chain structures derived from an insect accounted for about 6%, and was a very little. The sugar chain in which at least one GlcNAc was linked to M3 was detected and accounted for about 40%. Among them, the sugar chain to which two GlcNAc were linked (GN2) accounted for 23%. In addition, a portion also included a high-mannose type structure (FIG. 8). Besides, although the sugar chain to which a trace amount of fucose was linked was shown in FIG. 8, this was probably due to the contamination of components derived from tissues different from the middle silk gland. In addition, the designation regarding M6 in FIG. 8 meant that the sugar chain containing six mannoses accounted for almost all of them, and there were a possibility that the portion contained GN2 having fucose. In contrast, the fat body did not contain the addition of GlcNAc, and had a large amount of the structure such as M3 and FM2. In respect to these points, the fat body was significantly different from the middle silk gland. The sugar chain structure seen in the fat body had an agreement with the sugar chain structure of an insect, which had been known previously. The cocoon of the pnd-w1 strain had a little M3 as the middle silk gland did, the structure (GN2) to which two GlcNAc were linked accounted for about 40%, and was markedly abundant (FIG. 10). A similar analysis applied to Kinshu. However, there was no big difference in the sugar chain structure for the tissues and the cocoon when compared with pnd-w1, and there was no difference between the varieties (FIG. 9, FIG. 11, and FIG. 13).

In view of the above, the reason why the sugar chain structure of the murine IgG purified from a cocoon was different from the insect-specific sugar chain structure that had been previously reported was revealed to be due to the specificity of the tissue (silk gland) synthesizing the murine IgG.

In a baculovirus expression system, a recombinant protein was expressed by infecting a fat body with the virus. In contrast, the technique of the present inventors used a middle silk gland as a tissue for expressing a recombinant protein. The expression in this middle silk gland allowed the sugar chain without the addition of fucose and having a little paucimannose to be linked to a protein. This meant a significant advantage when the object of the production of the recombinant protein in the middle silk gland was directed to the addition of a human-type (a mammalian-type) sugar chain.

### Example 8

Generation of a transgenic Silkworm Expressing β-1,4-galactosyltransferase 1 and human β-1,2-N-acetylglucosaminyltransferase 2

cDNAs encoding β-1,4-galactosyltransferase 1 (hereinafter, referred to as hGalT1) and human β-1,2-N-acetylglucosaminyltransferase 2 (hereinafter, referred to as hGnT2) were cloned.

mRNA was extracted from human fibroblasts, and the cDNA was synthesized by using reverse transcriptase and oligo (dT) primers. The cDNA encoding each ORF of hGalT1 and hGnT2 was obtained by PCR using the synthesized cDNA as a template.

The primer sequences used to amplify the cDNA of hGalT1 were 5′-ATGAGGCTTCGGGAGCCGCT-3′ (SEQ ID NO: 9) and 5′-CTAGCTCGGTGTCCCGATGTCCA-3′ (SEQ ID NO: 10), and the primer sequences used to amplify the cDNA of hGnT2 were 5′-ATGAGGTTCCGCATCTACAAA-3′ (SEQ ID NO: 11) and 5′-TCACTGCAGTCTTCTATAACTTTTACAGAG-3′ (SEQ ID NO: 12).

The resulting PCR products were cloned into pCR-blunt2-TOPO (Invitrogen, Inc.). The nucleotide sequences were analyzed by a DNA sequencer, and the PCR products were verified to be the cDNAs encoding hGalT1 and hGnT2. The vectors containing hGalT1 and hGnT2 cDNAs were designated hereinafter as hGalT1/pCR2 and hGnT2/pCR2, respectively.

pMSG1.1R as described in Example 2 had a sericin promoter and a fibroin L-chain poly(A) addition sequence, and was designed to allow the cDNA of a desired protein to be inserted therebetween. In this experiment, in order to express two genes simultaneously, an additional promoter-poly(A) cassette was integrated into the AscI restriction enzyme site of pMSG1.1R to construct a new vector (pMSG3.1R).

In order to increase translation efficiency of hGalT1 and hGnT2, the polyhedrin 5′-untranslated region (hereinafter, referred to as 5′-UTR; sequence: AAGTATTTTACTGTTTTCGTAACAGTTTTGTAATAAAAAAACCTATAAAT (SEQ ID NO: 13)) derived from Bombyx mori nuclear polyhedrosis virus (hereinafter, referred to as BmNPV) was added to 5′-end of hGalT1 and hGnT2 cDNAs. Further, in order to add the restriction enzyme recognition sequence of NruI to both the 5′- and 3′-ends of the cDNAs, PCR was performed using the primers described below.

The primer sequences for hGalT1 were two kinds (5′-CACCTCGCGAAAGTATTTTACTGTTTTCGTAACAGTTTTGTAATAAAAAAA CCTATAAATATGAAGTTTCGGGAGCCGCTC-3′ (SEQ ID NO: 14) and 5′-TCGCGATTAGCTCGGCGTCCCGATGTC-3′ (SEQ ID NO: 15)). The primer sequences for hGnT2 were two kinds (5′-CACCTCGCGAAAGTATTTTACTGTTTTCGTAACAGTTTTGTAATAAAAAAA CCTATAAATATGAGGTTCCGCATCTACA-3′ (SEQ ID NO: 16) and 5′-TCGCGATTACTGCAGTCTTCTATAACTTTTACAGAGT-3′ (SEQ ID NO: 17)).

DNA fragments were each amplified by a PCR method using hGalT1/pCR2 and hGnT2/pCR2 as a template. The amplified products were cloned into a cloning vector, pCR-blunt2-TOPO (Invitrogen, Inc.), and the resulting vectors were designated as UTR-hGalT1/pCR2, and UTR-hGnT2/pCR2.

UTR-hGnT2/pCR2 was digested with restriction enzyme NruI, and the resulting DNA fragment of UTR-hGnT2 was inserted into the restriction enzyme Eco47III site of pMSG3.1MG. Next, the UTR-hGalT1 fragment obtained by digesting UTR-GalT1/pCR2 with NruI was inserted into the NruI site of the vector inserting UTR-hGnT2. Then, the correct nucleotide sequence and insertion orientation were verified. This vector was designated as hGalT1-hGnT2/pMSG3.1R (FIG. 14).

FIG. 14 showed a structure for generating a transgenic silkworm expressing hGalT and hGnT2 in a middle silk gland. In

FIG. 14, the meanings of the respective abbreviations were as follows:
piggyBacR: 3′-end sequence of piggyBac;
3xP3-TATA: a promoter inducing expression in the eyes and the nervous system;
DsRed: a red fluorescent protein gene;
SV40 polyA: a SV40-derived poly(A) addition signal;
Pser1: a silkworm sericin 1 gene promoter;
HR3: BmNP hr3;
hGalT1: a gene of human β-1,4-galactosyltransferase;
hGnT2: a gene of human β-1,2-N-acetylgulcosamiryltransferase;
FibL polyA; a silkworm fibroin L-chain poly(A) addition signal;
piggyBacL: 5′-end sequence of piggyBac;
Amp: an ampicillin antibiotic resistance gene

Besides, in the DNA sequence of this vector, the DNA sequence encoding β-galactosyltransferase is placed downstream of the DNA sequence encoding a sericin promoter. Accordingly, this vector is considered to be used to express β-galactosyltransferase in a middle silk gland. According to the Examples described below, when a glycoprotein is further expressed in the middle silk gland and is secreted to a sericin layer, β-galactose can be linked to the sugar chain of the glycoprotein, and the glycoprotein having a more humanized sugar chain can be produced. Similarly, in the DNA sequence of this vector, the DNA sequence encoding N-acetylglucosaminyltransferase is placed downstream of the DNA sequence encoding a sericin promoter. Accordingly, this vector is considered to be used to express N-acetylglucosaminyltransferase in a middle silk gland. According to the Examples described below, when the glycoprotein is further expressed in the middle silk gland and is secreted to a sericin layer, N-acetylglucosamine can be linked to the sugar chain of the glycoprotein, and the glycoprotein having a more humanized sugar chain can be produced.

### Example 9

Generation of a Transgenic Silkworm Expressing hGalT1 and hGnT2

hGalT1-hGnT2/pMSG3.1R was microinjected into total 2,984 eggs by a method similar to the method described in Example 3.

When the eggs microinjected with the DNA were incubated at 25° C., 819 eggs each hatched. Newly hatched silkworm continued to be raised, and the resulting reproductive imagos were crossed to yield 188 groups of F1 egg masses. The F1 egg masses at Day 5 to 6 after the spawning date were observed with a fluorescent stereomicroscope, eggs of a transgenic silkworm that emits red fluorescence from the eyes and the nervous system were screened. As a result, the egg masses containing the eggs of the transgenic silkworm (15 groups) were obtained. The resulting egg masses were subjected to hatching, and were raised. The transgenic silkworms (hereinafter, referred to as GalT-GnT2-TG silkworm) derived from 14 groups of the egg masses built a normal cocoon, became a pupa, and further emerged from the pupa to become a reproductive imago. Then, these silkworms were crossed with a wild-type silkworm to establish GalT-GnT2-TG silkworm.

### Example 10

Analysis of the Sugar Chain Structure of a Cocoon from GalT-GnT2-TG Silkworm

The sugar chain structure contained in a cocoon from a wild-type silkworm and GalT-GnT2-TG silkworm was analyzed by the method indicated in Example 6.

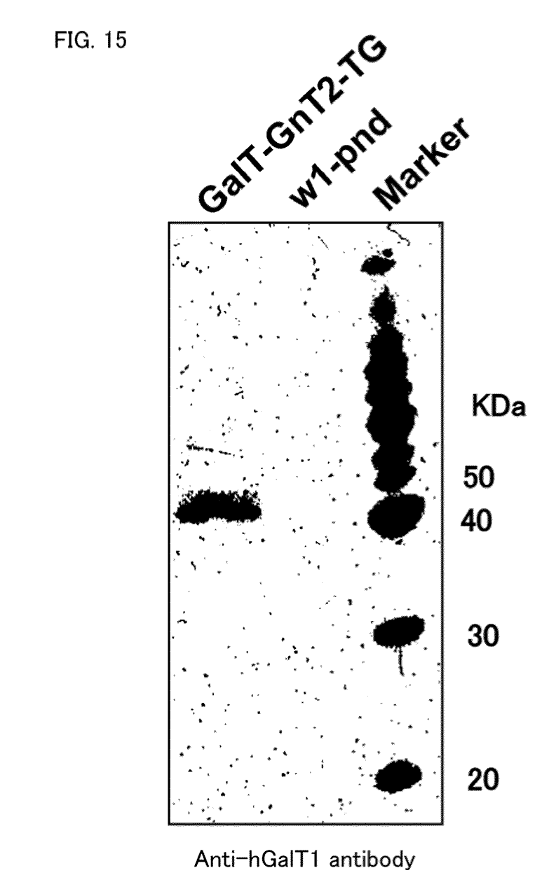

Among eight strains of GalT-GnT2-TG silkworm established, the sugar chain structural analysis of three strains was carried out. The strain names used in this analysis were referred to as 17-2, 75-1, and 65-4. Prior to the sugar chain structural analysis, the expression of hGalT1 in the middle silk gland of these three strains of the transgenic silkworm were analyzed by Western blotting method using an anti-hGalT1 antibody (the method was described in Examples, etc. of JP-A-2006-109772). Prior to the sugar chain structural analysis, the expression of hGalT1 in the middle silk gland of the GalT-GnT2-TG transgenic silkworm was analyzed by Western blotting method using an anti-hGalT1 antibody (the method was described in Examples, etc. of JP-A-2006-109772). As a result, the expression of hGalT1 was verified as shown in FIG. 15.

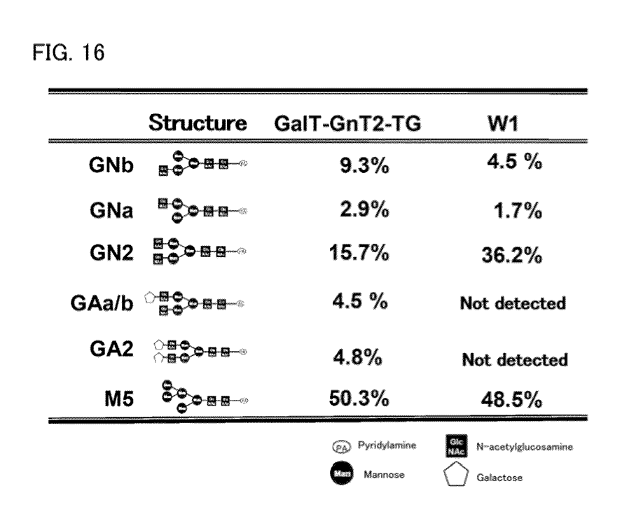

FIG. 16 showed the results obtained by the sugar chain structural analysis of a wild-type silkworm and GalT-GnT2-TG silkworm. The numerical values designated as % in the figure denoted the percentage (%) of the presence ratio calculated from the total sugar chain number of moles of the major peaks collected by reversed-phase HPLC.

The sugar chain having a structure to which at least one β-galactose (hereinafter, referred to as Gal) is linked was 0% for the cocoon from the wild-type silkworm. In contrast, it accounted for 9.3% (GAa/b was 4.5%, and GA2 was 4.8%) in the GalT-GnT2-TG silkworm. This demonstrated that the humanized sugar chain to which Gal was linked was linked in the middle silk gland of the transgenic silkworm expressing hGalT1 gene, which is β-galactosyltransferase.

### Example 11

In the GalT-GnT2-Tg silkworm, IgG was further expressed in the middle silk gland, and the experiment analyzing the sugar chain structure of the IgG was carried out. The results showed a success in expressing the IgG in the middle silk gland of the silkworm and in further expressing β-galactosyltransferase as well. For the results obtained by analyzing the sugar chain structure of this IgG, the results similar to the results obtained by analyzing the sugar chain structure of the cocoon from the GalT-GnT2-Tg silkworm described in Example 10 were obtained.

### Consideration of the Experimental Results

The results obtained by analyzing the sugar chain composition in total silkworm proteins, total silkworm proteins from the fat body, and total silkworm proteins from the silk gland tissue show a clear superiority of the silkworm silk gland in the humanization of the sugar chain when compared with the sugar chain composition of the glycoprotein such as IgG to begin with in CHO cells, which composition obtained from publications

That is, the humanization of an insect sugar chain requires two kinds of technological development (one is elongation of a sugar chain, and the other is removal of α-1,3-fucose). The removal of α-1,3-fucose is an unexpected result which has been revealed for the first time by the experiments of the present inventors. It is evident from the comparison of the experimental results described above that the sugar chain of the glycoprotein synthesized in the silk gland of a silkworm naturally has a little addition of α-1,3-fucose.

In addition, although the results are an unexpected result revealed for the first time by the experiments of the present inventors similarly, the comparison with the above experimental results clearly revealed that in the sugar chain of the glycoprotein naturally synthesized in the silk gland of a silkworm, the glycoprotein having the N-glycoside-linked sugar chain containing the sugar chain structure such as the abbreviations GNb, GNa, and GN2 in FIG. 8 to FIG. 16 (i.e., in other words, the glycoprotein having the N-glycoside-linked sugar chain containing the sugar chain structure represented by the above formulae (2), (3), and (4)) contains 20% or more.

In contrast, previous findings teach that the sugar chain having three or two mannoses (e.g., the sugar chain such as the abbreviations M3 and M2 in FIG. 8 to FIG. 16) accounts for almost all of the sugar chain synthesized in an insect. However, as revealed by the experiments at this time, for IgG purified from a cocoon of a silkworm and the sugar chain of the glycoprotein contained in the cocoon and the silk gland, the sugar chain having five mannoses (e.g., the abbreviation M5 in FIG. 8 to FIG. 16) and the sugar chain with GlcNAc (e.g., the abbreviations GNb, GNa, and GN2 in FIG. 8 to FIG. 16) are found abundant.

Here, the control CHO data used as what is called a positive control is the analysis results of IgG (i.e., as illustrated by using the abbreviations in the above FIG. 8 to FIG. 16, GN2 is about 51%, GNa+GNb is about 1%, M5 is about 3%, GA2 is 37%, GAa/b is 7%, M3 is 0%, and M2 is 0%), which are cited from a previously to known publication (Satoru KAMODA et al., “Capillary electrophoresis with laser-induced fluorescence detection for detailed studies on N-linked oligosaccharide profile of therapeutic recombinant monoclonal antibodies”, Journal of Chromatography A, 1133 (2006) 332-339). Other results are the analysis results of all the sugar chains that the present inventors indeed have cleaved from the cocoon and the tissue of a silkworm (in respect to GalT-GnT2-TG silkworm, those already generated in accordance with the above description have been used).

In the total silkworm proteins from the fat body, which is a major tissue for a previously known baculovirus expression system, the predicted results having a perfect agreement with the previous findings (e.g., the abbreviations M3 and M2 in FIG. 8 to FIG. 16 account for the almost all) have been obtained. In contrast, in the total proteins from the cocoon and the silk gland of a silkworm, the results similar to the data of IgG in the above silk gland (e.g., the abbreviations M5, GN2, GNa, and GNb in FIG. 8 to FIG. 16 are abundant) have been obtained. They are more similar to the sugar chains of CHO cells, which are a mammalian cell, (a mammalian type, a human type) than those of the fat body of a silkworm.

As described above, the sugar chain of the glycoprotein synthesized in the silk gland of an silkworm and secreted to the cocoon is found to have a strong advantage in respect to the humanization of the sugar chain (elongation of the sugar chain, without α-1,3-fucose). In addition, in order to improve the ADCC activity for the production of an antibody in the silk gland of a silkworm, the removal of α-1,6-fucose is necessary. However, in respect to this point, the following is an unexpected result which has been revealed for the first time by the experiments of the present inventors. It is evident from the comparison of the experimental results described above that the sugar chain of the glycoprotein synthesized in the silk gland of a silkworm naturally has no presence of α-1,6-fucose. That is, in the sugar chain of the glycoprotein synthesized in the silk gland of an silkworm and secreted to the cocoon, α-1,3-fucose is removed, α-1,6-fucose is also removed, and the sugar chain is elongated. Hence, these features have a strong advantage in the humanization of the sugar chain. It is further evident that they have also an advantage in the improvement of the ADCC activity for the production of an antibody.

In addition, the present inventors have generated a transgenic silkworm expressing β-galactosyltransferase (GalT) and N-acetylglucosaminyltransferase (GnT) in the silk gland, and have analyzed the sugar chain of the proteins of the cocoon. As described in the comparison of the above experimental results, the results are yet to be improved in respect to the ratio, but have shown a success in producing the sugar chain in which β-Gal is linked to the end of GlcNAc. This almost has accomplished the humanization (becoming a mammalian type) of a sugar chain. As described above, the humanization of a sugar chain (elongation of the sugar chain and removal of α-1,3-fucose) is possible by expressing only several glycosyltransferases together with a protein. Furthermore, even if no genetic modification (removal of α-1,6-fucose) is performed, the improvement of the ADCC activity is possible. Accordingly, it is said that there is a tremendous merit to use the silk gland of a silkworm as a tissue expressing a glycoprotein.

As described above, the present invention has been illustrated on the basis of Examples. These Examples are a just example, and it will be understood by those skilled in the art that a variety of modifications are possible, and that such modifications are within the scope of the present invention.

### INDUSTRIAL APPLICABILITY

As described in detail above, there is provided a glycoprotein having an N-glycoside-linked sugar chain in which fucose is not linked to N-acetylglucosamine at an reducing terminal of a sugar chain by using a silk gland cell (a middle silk gland cell) of an insect (a silkworm) classified as Lepidoptera or a silk gland itself of the insect body. Because of this, an antibody improving the ADCC activity and/or a glycoprotein (including an antibody) in which the antigenicity is decreased can be produced economically and in a large quantity. Therefore, it allows the economical and large production of a source material for an antibody medicine and a biopharmaceutical that is used in a variety of therapy to be achieved.

1. A method for producing a glycoprotein, comprising the step of expressing, in a silk gland cell of an insect, a glycoprotein having an N-glycoside-linked sugar chain in which fucose is not linked to N-acetylglucosamine at a reducing terminal of the sugar chain.

2. The method according to claim 1, wherein the insect is an insect classified into Lepidoptera.

3. The method according to claim 1, wherein the insect is a silkworm.

4. The method according to claim 1, wherein the silk gland cell is a middle silk gland cell.

5. The method according to claim 1, wherein the glycoprotein comprises a glycoprotein having an N-glycoside-linked sugar chain comprising a sugar chain structure represented by the formula (1):

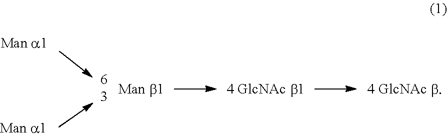

6. The method according to claim 1, wherein the glycoprotein comprises a glycoprotein having an N-glycoside-linked sugar chain comprising a sugar chain structure represented by the formula (2): 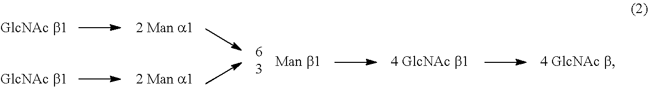
formula (3): 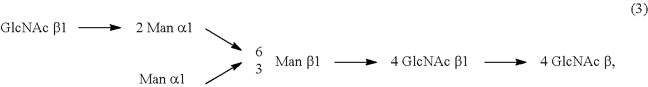
or formula (4):

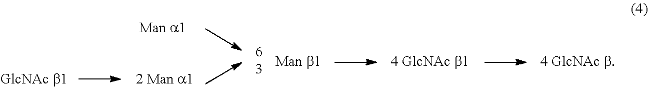

7. The method according to claim 1, wherein the glycoprotein is an antibody having a sugar chain.

8. The method according to claim 1, wherein among the glycoproteins, a ratio of a glycoprotein having a sugar chain structure in which α-1,6-fucose is linked to N-acetylglucosamine at a reducing terminal of an N-glycoside-linked sugar chain is 5% or less.

9. The method according to claim 8, wherein the glycoprotein is an antibody having a sugar chain; and
the antibody has an increased antibody-dependent cellular cytotoxic activity when compared with the same kind of an antibody having a typical sugar chain structure in a mammalian cell.

10. The method according to claim 1, wherein among all proteins produced, a ratio of a glycoprotein having a sugar chain structure in which α-1,3-fucose is linked to N-acetylglucosamine at a reducing terminal of an N-glycoside-linked sugar chain is 5% or less.

11. The method according to claim 10, wherein the glycoprotein is an antibody having a sugar chain: and
the antibody have reduced antigenicity in vivo in a mammal when compared with the same kind of an antibody having a typical sugar chain structure in an insect cell.

12. The method according to claim 1, wherein the silk gland cell is transformed to further increase an expression of N-acetylglucosaminyltransferase or β-galactosyltransferase.

13. The method according to claim 1, wherein the silk gland cell is transformed to increase an expression of β-galactosyltransferase; and
the glycoprotein comprises a glycoprotein having an N-glycoside-linked sugar chain comprising a sugar chain structure represented by the formula (5): 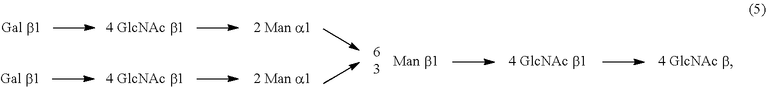
formula (6): 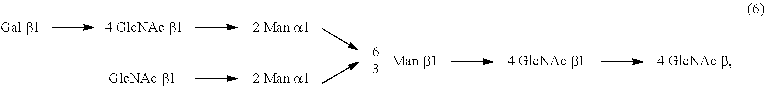
or formula (7):

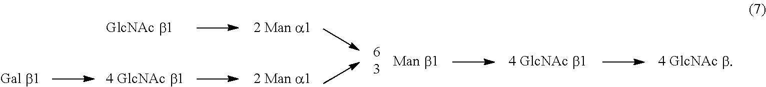

14. (canceled)

15. The method according to claim 13, wherein the glycoprotein contains 4% or more of a glycoprotein having an N-glycoside-linked sugar chain comprising a sugar chain structure represented by the formula (5), (6), or (7).

16. (canceled)

17. The method according to claim 1, wherein the step of expressing the glycoprotein comprises a step of accumulating a component comprising the glycoprotein in a cocoon in a silk gland of an insect body which is genetically modified to express the glycoprotein.

18. The method according to claim 17, wherein the glycoprotein is an antibody having a sugar chain; and
the step of expressing the glycoprotein comprises a step of using the insect body which is modified to coexpress an antibody heavy chain and an antibody light chain constituting the antibody as the insect body.

19. The method according to claim 17, wherein the glycoprotein is an antibody having a sugar chain; and
the step of expressing the glycoprotein comprises the steps of:
using an insect body in which both a gene encoding an antibody heavy chain and a gene encoding an antibody light chain are placed expressably downstream to a sericin promoter in a genome of the insect as the insect body; and
producing a cocoon containing the antibody in a sericin portion as the cocoon.

20-22. (canceled)

23. A glycoprotein-producing cell, comprising a cell producing a glycoprotein having an N-glycoside-linked sugar chain in which fucose is not linked to N-acetylglucosamine at a reducing terminal of the sugar chain, the cell being a silk gland cell of an insect genetically modified to express the glycoprotein.

24. The cell according to claim 23, wherein the silk gland cell is transformed to increase an expression of N-acetylglucosaminyltransferase.

25. The cell according to claim 23, wherein the silk gland cell is transformed to increase an expression of β-galactosyltransferase.

26. A glycoprotein-producing organism, comprising an organism producing a glycoprotein having an N-glycoside-linked sugar chain in which fucose is not linked to N-acetylglucosamine at a reducing terminal of the sugar chain, the organism comprising an insect body genetically modified to express the glycoprotein in a silk gland.

27. The organism according to claim 26, wherein the insect body is transformed to increase an expression of N-acetylglucosaminyltransferase in the silk gland.

28. The organism according to claim 26, wherein the insect body is transformed to increase an expression of β-galactosyltransferase in the silk gland.

29. A DNA construct for adding β-galactose to a sugar chain of a glycoprotein, comprising a DNA sequence in which a DNA sequence encoding β-galactosyltransferase is operably linked to a DNA sequence encoding a sericin promoter.

30. A DNA construct for adding N-acetylglucosamine to a sugar chain of a glycoprotein, comprising a DNA sequence in which a DNA sequence encoding N-acetylglucosaminyltransferase is operably linked to a DNA sequence encoding a sericin promoter.

31. A DNA construct for humanizing a sugar chain structure of a glycoprotein, wherein the DNA construct according to claim 29 further comprises a DNA sequence encoding N-acetylglucosaminyltransferase.

32. The method according to claim 1, wherein the silk gland cell is transformed to increase expressions of both N-acetylglucosaminyltransferase and β-galactosyltransferase.
